SEQUENCE LISTING (3 sequences)

SEQ ID NO: 1 - DNA, 5070 bp, Homo sapiens
cgcgacctcagatcagacgtggcgacccgctgaatttaagcatattagtcagcggaggagaagaaactaaccaggattccctcagtaacggcgagtgaacagggaagagcccagcgccgaatccccgccccgcggcggggcgcgggacatgtggcgtacggaagacccgctccccggcgccgctcgtggggggcccaagtccttctgatcgaggcccagcccgtggacggtgtgaggccggtagcggcccccggcgcgccgggcccgggtcttcccggagtcgggttgcttgggaatgcagcccaaagcgggtggtaaactccatctaaggctaaataccggcacgagaccgatagtcaacaagtaccgtaagggaaagttgaaaagaactttgaagagagagttcaagagggcgtgaaaccgttaagaggtaaacgggtggggtccgcgcagtccgcccggaggattcaacccggcggcgggtccggccgtgtcggcggcccggcggatctttcccgccccccgttcctcccgacccctccacccgccctcccttcccccgccgcccctcctcctcctccccggagggggcgggctccggcgggtgcgggggtgggcgggcggggccgggggtggggtcggcgggggaccgtcccccgaccggcgaccggccgccgccgggcgcatttccaccgcggcggtgcgccgcgaccggctccgggacggctgggaaggcccggcggggaaggtggctcggggggccccgtccgtccgtccgtccgtcctcctcctcccccgtctccgccccccggccccgcgtcctccctcgggagggcgcgcgggtcggggcggcggcggcggcggcggtggcggcggcggcggcggcggcgggaccgaaaccccccccgagtgttacagcccccccggcagcagcactcgccgaatcccggggccgagggagcgagacccgtcgccgcgctctcccccctcccggcgcccacccccgcggggaatcccccgcgaggggggtctcccccgcgggggcgcgccggcgtctcctcgtgggggggccgggccacccctcccacggcgcgaccgctctcccacccctcctccccgcgcccccgccccggcgacggggggggtgccgcgcgcgggtcggggggcggggcggactgtccccagtgcgccccgggcgggtcgcgccgtcgggcccgggggaggttctctcggggccacgcgcgcgtcccccgaagagggggacggcggagcgagcgcacggggtcggcggcgacgtcggctacccacccgacccgtcttgaaacacggaccaaggagtctaacacgtgcgcgagtcgggggctcgcacgaaagccgccgtggcgcaatgaaggtgaaggccggcgcgctcgccggccgaggtgggatcccgaggcctctccagtccgccgagggcgcaccaccggcccgtctcgcccgccgcgccggggaggtggagcacgagcgcacgtgttaggacccgaaagatggtgaactatgcctgggcagggcgaagccagaggaaactctggtggaggtccgtagcggtcctgacgtgcaaatcggtcgtccgacctgggtataggggcgaaagactaatcgaaccatctagtagctggttccctccgaagtttccctcaggatagctggcgctctcgcagacccgacgcacccccgccacgcagttttatccggtaaagcgaatgattagaggtcttggggccgaaacgatctcaacctattctcaaactttaaatgggtaagaagcccggctcgctggcgtggagccgggcgtggaatgcgagtgcctagtgggccacttttggtaagcagaactggcgctgcgggatgaaccgaacgccgggttaaggcgcccgatgccgacgctcatcagaccccagaaaaggtgttggttgatatagacagcaggacggtggccatggaagtcggaatccgctaaggagtgtgtaacaactcacctgccgaatcaactagccctgaaaatggatggcgctggagcgtcgggcccatacccggccgtcgccggcagtcgagagtggacgggagcggcgggggcggcgcgcgcgcgcgcgcgtgtggtgtgcgtcggagggcggcggcggcggcggcggcgggggtgtggggtccttcccccgcccccccccccacgcctcctcccctcctcccgcccacgccccgctccccgcccccggagccccgcggacgctacgccgcgacgagtaggagggccgctgcggtgagccttgaagcctagggcgcgggcccgggtggagccgccgcaggtgcagatcttggtggtagtagcaaatattcaaacgagaactttgaaggccgaagtggagaagggttccatgtgaacagcagttgaacatgggtcagtcggtcctgagagatgggcgagcgccgttccgaagggacgggcgatggcctccgttgccctcggccgatcgaaagggagtcgggttcagatccccgaatccggagtggcggagatgggcgccgcgaggcgtccagtgcggtaacgcgaccgatcccggagaagccggcgggagccccggggagagttctcttttctttgtgaagggcagggcgccctggaatgggttcgccccgagagaggggcccgtgccttggaaagcgtcgcggttccggcggcgtccggtgagctctcgctggcccttgaaaatccgggggagagggtgtaaatctcgcgccgggccgtacccatatccgcagcaggtctccaaggtgaacagcctctggcatgttggaacaatgtaggtaagggaagtcggcaagccggatccgtaacttcgggataaggattggctctaagggctgggtcggtcgggctggggcgcgaagcggggctgggcgcgcgccgcggctggacgaggcgccgccgccccccccacgcccggggcacccccctcgcggccctcccccgccccaccccgcgcgcgccgctcgctccctccccgccccgcgccctctctctctctctctcccccgctccccgtcctcccccctccccgggggagcgccgcgtgggggcggcggcggggggagaagggtcggggcggcaggggccggcggcggcccgccgcggggccccggcggcgggggcacggtcccccgcgaggggggcccgggcacccggggggccggcggcggcggcgactctggacgcgagccgggcccttcccgtggatcgccccagctgcggcgggcgtcgcggccgcccccggggagcccggcgggcgccggcgcgcccccccccccaccccacgtctcgtcgcgcgcgcgtccgctgggggcggggagcggtcgggcggcggcggtcggcgggcggcggggcggggcggttcgtccccccgccctacccccccggccccgtccgccccccgttcccccctcctcctcggcgcgcggcggcggcggcggcaggcggcggaggggccgcgggccggtcccccccgccgggtccgcccccggggccgcggttccgcgcggcgcctcgcctcggccggcgcctagcagccgacttagaactggtgcggaccaggggaatccgactgtttaattaaaacaaagcatcgcgaaggcccgcggcgggtgttgacgcgatgtgatttctgcccagtgctctgaatgtcaaagtgaagaaattcaatgaagcgcgggtaaacggcgggagtaactatgactctcttaaggtagccaaatgcctcgtcatctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccggcgcccccccggtgtccccgcgaggggcccggggcggggtccgccggccctgcgggccgccggtgaaataccactactctgatcgttttttcactgacccggtgaggcgggggggcgagccccgaggggctctcgcttctggcgccaagcgcccggccgcgcgccggccgggcgcgacccgctccggggacagtgccaggtggggagtttgactggggcggtacacctgtcaaacggtaacgcaggtgtcctaaggcgagctcagggaggacagaaacctcccgtggagcagaagggcaaaagctcgcttgatcttgattttcagtacgaatacagaccgtgaaagcggggcctcacgatccttctgaccttttgggttttaagcaggaggtgtcagaaaagttaccacagggataactggcttgtggcggccaagcgttcatagcgacgtcgctttttgatccttcgatgtcggctcttcctatcattgtgaagcagaattcaccaagcgttggattgttcacccactaatagggaacgtgagctgggtttagaccgtcgtgagacaggttagttttaccctactgatgatgtgttgttgccatggtaatcctgctcagtacgagaggaaccgcaggttcagacatttggtgtatgtgcttggctgaggagccaatggggcgaagctaccatctgtgggattatgactgaacgcctctaagtcagaatcccgcccaggcggaacgatacggcagcgccgcggagcctcggttggcctcggatagccggtcccccgcctgtccccgccggcgggccgccccccccctccacgcgccccgcgcgcgcgggagggcgcgtgccccgccgcgcgccgggaccggggtccggtgcggagtgcccttcgtcctgggaaacggggcgcggccggagaggcggccgccccctcgcccgtcacgcaccgcacgttcgtggggaacctggcgctaaaccattcgtagacgacctgcttctgggtcggggtttcgtacgtagcagagcagctccctcgctgcgatctattgaaagtcagccctcgacacaagggtttgtc

SEQ ID NO: 2 - DNA, 1869 bp, Homo sapiens
tacctggttgatcctgccagtagcatatgcttgtctcaaagattaagccatgcatgtctgagtacgcacggccggtacagtgaaactgcgaatggctcattaaatcagttatggttcctttggtcgctcgctcctctcctacttggataactgtggtaattctagagctaatacatgccgacgggcgctgacccccttcgcgggggggatgcgtgcatttatcagatcaaaaccaacccggtcagcccctctccggccccggccggggggcgggcgccggcggctttggtgactctagataacctcgggccgatcgcacgccccccgtggcggcgacgacccattcgaacgtctgccctatcaactttcgatggtagtcgccgtgcctaccatggtgaccacgggtgacggggaatcagggttcgattccggagagggagcctgagaaacggctaccacatccaaggaaggcagcaggcgcgcaaattacccactcccgacccggggaggtagtgacgaaaaataacaatacaggactctttcgaggccctgtaattggaatgagtccactttaaatcctttaacgaggatccattggagggcaagtctggtgccagcagccgcggtaattccagctccaatagcgtatattaaagttgctgcagttaaaaagctcgtagttggatcttgggagcgggcgggcggtccgccgcgaggcgagccaccgcccgtccccgccccttgcctctcggcgccccctcgatgctcttagctgagtgtcccgcggggcccgaagcgtttactttgaaaaaattagagtgttcaaagcaggcccgagccgcctggataccgcagctaggaataatggaataggaccgcggttctattttgttggttttcggaactgaggccatgattaagagggacggccgggggcattcgtattgcgccgctagaggtgaaattcttggaccggcgcaagacggaccagagcgaaagcatttgccaagaatgttttcattaatcaagaacgaaagtcggaggttcgaagacgatcagataccgtcgtagttccgaccataaacgatgccgaccggcgatgcggcggcgttattcccatgacccgccgggcagcttccgggaaaccaaagtctttgggttccggggggagtatggttgcaaagctgaaacttaaaggaattgacggaagggcaccaccaggagtggagcctgcggcttaatttgactcaacacgggaaacctcacccggcccggacacggacaggattgacagattgatagctctttctcgattccgtgggtggtggtgcatggccgttcttagttggtggagcgatttgtctggttaattccgataacgaacgagactctggcatgctaactagttacgcgacccccgagcggtcggcgtcccccaacttcttagagggacaagtggcgttcagccacccgagattgagcaataacaggtctgtgatgcccttagatgtccggggctgcacgcgcgctacactgactggctcagcgtgtgcctaccctacgccggcaggcgcgggtaacccgttgaaccccattcgtgatggggatcggggattgcaattattccccatgaacgaggaattcccagtaagtgcgggtcataagcttgcgttgattaagtccctgccctttgtacacaccgcccgtcgctactaccgattggatggtttagtgaggccctcggatcggccccgccggggtcggcccacggccctggcggagcgctgagaagacggtcgaacttgactatctagaggaagtaaaagtcgtaacaaggtttccgtaggtgaacctgcggaaggatcatta

SEQ ID NO: 3 - DNA, 157 bp, Homo sapiens
cgactcttagcggtggatcactcggctcgtgcgtcgatgaagaacgcagctagctgcgagaattaatgtgaattgcaggacacattgatcatcgacacttcgaacgcacttgcggccccgggttcctcccggggctacgcctgtctgagcgtcgctt

(19) United States
(12) Patent Application Publication
(10) Pub. No.: US 2024/0102100 A1
(43) Pub. Date: Mar. 28, 2024

(54) RIBOSOMAL RNAS 2'O-METHYLATION AS A NOVEL SOURCE OF BIOMARKERS RELEVANT FOR DIAGNOSIS, PROGNOSIS AND THERAPY OF CANCERS

(71) Applicants: CENTRE LEON BERARD, LYON (FR); CENTRE NATIONAL DE LA RECHERCHE SCIENTIFIQUE (CNRS), PARIS (FR); INSERM (Institut National de la Sante et de la Recherche Medicale), PARIS (FR); UNIVERSITE CLAUDE BERNARD LYON 1, VILLEURBANNE (FR)

(72) Inventors: Jean-Jacques DIAZ, VENISSIEUX (FR); Virginie MARCEL-TERRIER, LYON (FR); Janice KIELBASSA, COREAS (FR); Frédéric CATEZ, GENAS (FR)

(21) Appl. No.: 18/038,385

(22) PCT Filed: Nov. 29, 2021
(86) PCT No.: PCT/EP2021/083429

§ 371 Date: May 23, 2023

(30) Foreign Application Priority Data
Nov. 27, 2020 (EP) 20306461.3

(51) Int. Cl. C12Q 1/6886

(52) U.S. Cl. CPC C12Q 1/6886; C12Q 2600/106; C12Q 2600/118; C12Q 2600/154

(57) ABSTRACT

The present invention relates to a method for identifying potentially relevant markers in cancer diagnosis, prognosis and/or therapy, comprising an analysis approach which is based on the detection of variations in methylation of ribosomal RNAs in a biological sample. The present invention also relates to several applications of this analysis approach for determining the cancer subtype and/or the prognosis of a patient suffering from cancer, for estimating or assessing the benefit of a treatment in such patient, but also for selecting one or more therapeutic drug(s) targeting ribosomes useful for treating cancers.

### TECHNICAL FIELD OF THE INVENTION

The present invention belongs to the medical field, and in particular to the field of cancer biomarkers and therapies.

The present invention relates to a method for identifying potentially relevant markers in cancer diagnosis, prognosis and/or estimation of benefit of treatment and/or therapy, comprising an analytic approach which is based on the detection of variations in 2′O-ribose methylation of ribosomal RNAs (rRNAs) in a biological sample. The inventors indeed demonstrated that among such variations, some are relevant markers since associated with a clinical significance in cancer. The present invention also relates to several applications of this analysis approach for determining the prognosis of a patient suffering from cancer, for estimating or assessing the benefit of a treatment in such patient, but also for selecting one or more therapeutic drug(s) targeting ribosomes useful for treating cancers. Each of these applications may also have a diagnostic purpose, in particular by identifying molecular cancer sub-types, in some specific cases. The present invention also relates to kits comprising molecules able to specifically recognize the modified regions on the rRNAs containing the relevant markers which are accurately associated with a clinical significance in cancer, and uses thereof in diagnosis, prognosis, estimation of benefit of treatment and/or therapy; all of that preferably in case of a breast cancer and glioma.

### BACKGROUND TO THE INVENTION

Gene expression is a multi-step process that finely shapes the cellular phenotype. Until recently, mRNA synthesis from DNA that includes regulation of both chromatin accessibility (i.e., epigenetic) and transcription, remains the most studied step. However, it appears that translation of mRNA into protein can be also tightly controlled and directly contributes in acquisition of particular phenotype, including in cancer (Truitt and Ruggero, Nat Rev Cancer 2016). Among the different mechanisms regulating translation, growing evidence support the unexpected notion that the ribosome itself acts as a direct actor of translation. In particular, emerging evidence suggests that changes in ribosomal RNA 2′O-ribose methylation (rRNA 2′OMe, rRNA 2′Ome or rRNA 2′O-methylation thereafter) in the human ribosomes play a key role in regulating translation (i.e., rRNA epitranscriptomic) thereby contributing in setting particular phenotypes such as hallmarks of cancer cells (1-3).

Historically, plethora of studies mainly performed in both yeast and Xenopus laevis models, which have been the historical prototypic, eukaryotic models used during many years finely decipher the highly complex molecular mechanisms of rRNA transcription, chemical modifications and processing (4). These studies allowed the mapping of rRNA 2′O-methylation sites but also the demonstration of the importance of 2′O-methylation in the conserved intrinsic activity of the ribosome, on which life relies. Indeed, it appeared that loss of 2′O-methylation at a few numbers of rRNA sites in yeast is critical for ribosome functions and induces cell death (4-5). However, more recent findings indicate that in yeast, loss of rRNA 2′O-methylation at other sites have only little or no effect.

Indeed, it emerges from the last decade that rRNA 2′O-methylation is an additional layer of gene expression regulation, highlighting the ribosome as a novel actor of translation control. This new layer of gene expression regulation recently uncovered, joins the numerous descriptions of chemical modifications of both coding and non-coding RNAs that regulates post-transcriptional processes, a field known as epitranscriptomics (5-6).

To date, this major finding lies on evidences coming mainly, if not exclusively, from cellular models most of them representing either cancer cell lines or experimentally-modified in vitro models, the latter being established using genetic or molecular biology approaches for trying to mimic pre-cancerous stage, tumoral initiation or tumour progression (3, 5). Moreover, since the currently available technics used to be site-specific approaches, having a comprehensive overview of the variability of rRNA 2′O-methylation at all the positions in cellular models was difficult to obtain. Thus, at present, it is unclear whether rRNA 2′O-methylation varies in humans.

Although genetics, genomics, epigenetics, transcriptomics and proteomics revealed the heterogeneity of tumors at the molecular level, identification of novel molecular layers are required to fully capture all the inter-patient variability, essential for developing clinical applications. Indeed, all if not all these approaches concentrated their attention on transcription regulation. However, in several physio-pathological conditions including cancer, transcriptome is clearly different from the translatome, indicating that translational regulation plays a yet underestimated role in shaping cellular phenotype. Among these transcription-centered approaches, epigenetics appears as promising additional molecular fingerprints to improve diagnosis (i.e., patient classification), prognosis or treatment. Epigenetic mainly corresponds to base methylation of the cytosine nucleotide in DNA that can be added by different methyltransferases in a sequence-specific manner thanks to the cooperation with transcription factors, and that can be interpretated by additional proteins to regulate transcription. Due to the accessibility of -omic approaches dedicated to both transcriptomic and epigenetic, numerous studies demonstrated the importance of these two molecular mechanisms in shaping cellular phenotype. However, it also fails to illustrate all the heterogeneity of cancer phenotype by focusing only on transcription regulation. In the molecular portraits of cancers, RNA epitranscriptomics and in particular rRNA 2′O-methylation, has never been investigated.

### DETAILED DESCRIPTION OF THE INVENTION

Thanks to the recent resolution of technological issues, including the development of RNA-seq based approaches dedicated to the profiling of rRNA 2′O-methylation (3, 7-9), the inventors had achieved rRNA 2′O-methylation profiling in human samples to determine whether it can provide a molecular fingerprint in cancers. These data reveal the unexpected existence of specific modulation of 2′O-methylation at only particular rRNA positions between human tumors that are surprisingly associated with clinical outcome and biological features.

In particular, the first rRNA 2′O-methylation profiling of human breast tumors and gliomas was established, using the innovative RiboMeth-seq technology adapted to perform high-throughput analyses of human clinical samples.

The inventors uncovered the existence of stable sites, which show limited inter-patient variability in their 2′O-methylation level, which map on functionally important sites of the human ribosome structure. These stable sites are surrounded by variable sites, which map on the second nucleotide layer of the human ribosome structure. The inventors' data demonstrate that some positions within the rRNA molecules can tolerate absence of 2′O-methylation in tumoral and healthy human tissues. These data also reveal that rRNA 2′O-methylation exhibits intra- and inter-patient variability in different types of tumors, and particularly in breast and glioma tumors. rRNA 2′Ome level is indeed differentially associated with breast cancer and glioma subtype and tumor grade.

The present invention which provides rRNA 2′O-methylation profiling of large-scale human sample collections offers the first compelling evidence that ribosome variability occurs in humans and that rRNA 2′O-methylation represent a relevant element of tumour biology useful in clinic. The present invention based on this novel variability at molecular level offers an additional layer to capture the cancer heterogeneity and associates with specific features of tumour biology thus offering a novel targetable molecular signature in cancer.

With the present invention, the inventors have shown that rRNA 2′O-methylation-related signatures, corresponding to either a global 2′O-methylation signature based on all the 2′O-methylated rRNA sites or a site-specific signature whose source corresponds to only variable sites, respectively carry clinical information. The advantages in identifying individual or even unique sites of interest, allow the usage of less expensive techniques suitable for routine usage in clinic to determine the 2′O-methylation level at a particular site only for diagnosis, prognosis and/or therapeutic purposes.

Indeed, the inventors had shown for the first time that rRNA molecules exist naturally in humans as molecules not fully and equally 2′O-methylated at all the 2′O-methylation positions in humans. Using structure/function and evolution enrichment analyses as well as the differential association with biological characteristics, the inventors had demonstrated that at least two classes of rRNA 2′O-methylated sites concur in human cancers, depending on their ability to tolerate (i.e., variable sites) or not (i.e., stable sites) this chemical modification. This unique discovery made by comparing human samples demonstrates the co-existence of stable and variable 2′O-methylation at specific rRNA positions.

rRNA epitranscriptomics offers thus the opportunity to take into account an additional step in gene expression that displays its own specificities. In contrast to epigenetic, rRNA epitranscriptomic indeed mostly corresponds to ribose methylation of all sort of nucleotide in rRNA that is added for 95% of them by a unique methyltransferase, the fibrillarin (FBL), which is guided by non-coding RNAs (or snoRNA) in a sequence-dependent manner. Although it was though for a long time that presence of 2′O-methylation is compulsory on rRNA to shape its particular structure on which its activity relies, it surprisingly emerges that the presence or absence of rRNA 2′O-methylation can directly affect regulation of mRNA translation. In addition to affect its translational activity, by modifying its structure, presence or absence of 2′O-methylation on a single rRNA position might be sufficient to affect small molecule binding, such as antibiotic. Indeed, it has been shown that some resistance to antibiotic in bacteria relies on the appearance of 2′O-methylation at a novel rRNA position. Thus, presence or absence of rRNA 2′O-methylation at a position in the vicinity of binding region of molecules might drastically affect its efficacy. These observations support the notion that rRNA 2′O-methylation provides not only an additional layer of characterization of cancer reflecting alteration in translational regulation but also a rational to identify cancer ribosome-targeting molecules.

In a first aspect, the present invention therefore relates to a method for identifying potentially relevant markers in cancer diagnosis, prognosis and/or estimation of treatment benefit and/or therapy comprising:
a) measuring the 2′O-ribose methylation level of the 2′O-ribose methylation positions of ribosomal RNAs (rRNAs) in biological samples from a representative population of patients suffering from a cancer,
b) assessing the individual methylation status for each 2′O-ribose methylation positions by determining the variability of the 2′O-ribose methylation level thus measured for each 2′O-ribose methylation position between each sample of patients from the representative population,
c) selecting the set of 2′O-ribose methylation positions for which the individual methylation status is a “variable status”.

In particular, in the general context of the present invention, as well as for step b) defined above, assessment of the methylation status may be carried out by:
b1) determining the variability of the 2′O-ribose methylation level measured for each 2′O-ribose methylation position between each sample of patients from the representative population, and
b2) determining for each 2′O-ribose methylation position the methylation status by comparing the variability of all 2′O-ribose methylation positions among the representative population.

The method of the invention allows identification of a set of potentially relevant markers useful in clinic for management of patients suffering from cancer. This set corresponds to potentially relevant methylation positions in rRNAs to be analyzed and in which, by judicious comparison of the methylation status compared to that of a reference population, those accurately having clinical significance may be selected.

All the methods according to the invention described above are in vitro or ex vivo methods.

As used herein, the terms “tumours” or “tumor” refer to a malign tumor, so the terms “tumours”, “tumor” and “cancer” are used interchangeably and have the same definition.

The term “representative population” used herein is intended to mean a population of human patients summarizing the clinical characteristics of one particular cancer type. In all the applications methods of the present invention described here after, for diagnosis, for determining prognostic, for estimating benefit to a treatment, the “representative population” is selected to be adapted to the dedicated respective applications. For example, for diagnosis purpose, the “representative population” is composed of a population of patients for which the cancer subtypes is known. For example, for prognostic purpose, the “representative population” is composed of a population of patients for which the prognostic is known. For estimating benefit to a treatment, the “representative population” is composed of a population of patients for which the benefit of the said treatment is known. In these cases, the “representative population” is thus corresponding to a “reference population”. However, where the comparison to be performed is with a population of patients who do not suffer from a cancer, as in one of the embodiments of the methods of the invention for selecting therapeutic drug(s) targeting ribosomes, the “representative population” is rather corresponding to a “control population”.

As used in the present invention, the term “diagnostic” or “diagnosis” is limited to identification of cancer subtypes, that is to say the identification within a single cancer type coming from the same organ, of the different groups of tumors displaying similar anatomopathological or molecular traits indicative of common biological features. In particular, classification of tumors by their molecular cancer subtypes allows to distinguish tumors coming from the same organ but with intrinsic molecular particularities allowing to improve patient management by providing adapted therapeutic protocols.

Herein the terms “biological sample” and “sample” are used interchangeably. All types of biological samples may be used in the method of the invention. Preferably, the biological sample to be used in the method of the invention is selected among tissues (i.e., surgical piece, biopsies, Formalin Fixed Paraffin Embedded tissue) and biologic fluids (i.e., blood sample, sputum, urine, and the like), biopsies being the preferable biological sample to use.

Terms “2′O-ribose methylation”, “2′O-methylation”, “2′Ome”, “2′O-me”, “2′OMe”, “2′-O-Me” or “2′O-Me” are herein used interchangeably and have the same definition which refers to methylation at 2′O position of a ribose.

The words “site” and “position” are used interchangeably and have the same definition which refers to the nucleotide that carry the 2′O-methylation.

Ribosomal RNAs or rRNAs means the four ribosomal RNAs 5S, 5.8S, 18S and 28S. The total size of the four rRNAs is of 7.2 kb. The rRNAs 28S, 18S and 5.8S are generated after cleavage of a single pre-rRNA precursor.

Thus, the terms “methylation position”, “methylation site”, “2′O-ribose methylation position”, “2′O-ribose methylation site”, “2′Ome position”, “2′Ome site”, “2′OMe position”, “2′OMe site”, “methylated position” and “methylated site”, as used herein have the same definition and refer to all nucleotides of rRNAs at which the ribose may be methylated in 2′O.

According to the present invention, the terms “2′O-ribose methylation level”, “2′O-methylation level”, “methylation level”, “level of 2′O-ribose methylation”, “level of methylation” or “level of methylation” have the same significance and can be used interchangeably. All those terms refer to the determination of a relative measure.

The “level of methylation” of the different methylation sites of rRNAs, is to be detected by any relevant technology which is known in the art. In particular, the methylation level is determined by any method allowing to determine whether a 2′O position of a ribose is methylated or not. In a preferred embodiment, said methylation level is determined by non-sequencing methods (RNA fingerprinting, primer extension-based approach, mass spectrometry, RP-HPLC, X-ray crystallography and cryo-EM) and sequencing-based high-throughput methods (2OMe-seq, RimSeq, CLIP-seq, RibOxi-seq, Nm-seq, RiboMeth-seq) (3, 9).

In a preferred embodiment of the method of the invention, the 2′O-ribose methylation level of the 2′O-ribose methylation positions is determined by a C-score calculated using RiboMeth-seq method.

The RiboMeth-seq technique calculates a 2′Ome level for each of all the methylation positions individually. This level is called “C-score” and is calculated from the ratio of the 5′ and/or 3′ end reads raw count at the position n to the local read coverage, where the latter is inferred from the 5′ and/or 3′ end read raw counts of the flanking positions (i.e., k=x nucleotides located upstream and downstream from n) (8). It does not correspond to an absolute value and only allows a relative comparison:
If C-score=1 at position n, all rRNAs in the sample are 2′Ome at position n
If C-score=0 at position n, none of the rRNAs in the sample are 2′Ome at position n.
If C-score is between 0 and 1 at a position n, two types of rRNA are present in the sample, (1) rRNAs carrying a 2′Ome and (2) rRNAs not carrying the 2′Ome, at position n in varying proportions.

According to this embodiment, a dataset corresponding to the C-scores of the integrality of the methylation positions is therefore obtained for each sample, one C-score per 2′Ome site.

The general approach on which the present invention is founded is a comparison of the variability of the 2′Ome level, preferably the C-score, calculated at each of all the methylation sites with data from all available samples. The threshold for identifying “stable” and “variant” (or “variable”) sites corresponds to the variability value from which a deviation from the majority of all sites is observed.

The methylation status of each 2′O-ribose methylation position is determined regarding a threshold corresponding to the minimal value of the variability of the 2′O-ribose methylation level at one particular position, that shows a deviation from the variability of the 2′O-ribose methylation level of the other 2′O-ribose methylation positions among the representative population. This threshold thus allows to designate the methylation status, corresponding to either “stable” or “variable” status.

Therefore, in a preferred embodiment of the method of the invention, the variability of the 2′O-ribose methylation level for each 2′O-ribose methylation position, which is determined for assessing the methylation status, is determined by a statistical approach which allows the comparison of the variability of rRNA 2′Ome level between each sample of patients from the representative population for each 2′Ome position independently. Variability estimation includes usual statistical methods to indicate the dispersion of the 2′O-methylation level, such as range (maximum to minimum value difference), interquartile range (IQR), variance, standard deviation or quartile coefficient of dispersion.

Then the methylation status of each 2′O-ribose methylation position is determined regarding a threshold corresponding to the minimal value of the variability of the 2′O-ribose methylation level at one particular position, that shows a deviation from the values of the variability of the 2′O-ribose methylation level of the other 2′O-ribose methylation positions among the representative population. Deviation estimation includes usual mathematical and statistical methods, such as linear regression or straight line from ascending value of the variability estimator. In the latter example associated with the usage of IQR as variability estimator of 2′O-methylation level, the cut-off position was defined as the position where the increase of the IQR between two successive positions is not constant anymore. 2′O-methylated positions having IQR values below the threshold were termed “stable”, while positions having IQR values above were designated as “variable”.

Thus, the methylation status includes two possible statuses:
Stable status: where variability of 2′Ome at a particular site is below the threshold, as defined above, and
Variable status: where variability of 2′Ome at a particular site is above the threshold as defined above.

Distinction between those two statuses is by definition relative since it is a comparison between thereto. Preferably, this distinction between “stable” 2′Ome sites and “variable” 2′Ome sites is based on a statistical approach.

All sites with a variable status, also named herein as “variable site” or “variant site”, which constitute the set of markers potentially relevant in cancer prognosis and/or estimation of the treatment benefit and/or therapy are contains in rRNAs 28S, 18S, 5.8S. Similarly, all sites with a stable status, also named herein as “stable site”, are contains in rRNAs 28S, 18S, 5.8S. However, those sites have no clinical information, relevance or significance per se.

In this context, the method of the present invention thus allows the identification of “stable” 2′Ome sites, meaning the 2′Ome sites with a stable methylation status between patients, which do not carry biological/clinical information, and the identification of “variant” 2′Ome sites, meaning the 2′Ome sites with a variable methylation status between patients, carrying biological/clinical information.

### Information on 2′O-ribose Methylation (2′Ome)

106 2′Ome sites are known to be spread over 28S, 18S and 5.8S ribosomal RNAs only, corresponding to 106 nucleotides identified to date out of 7067 putative carriers of a 2′Ome.

Each of those 2′Ome position corresponding to 2′O-ribose methylation is identified according to a normalized method (11), based on the following information in this order:
Name of the rRNA (among 28S, 18S and 5.8S only)
Underscore or hyphen
Nature of the modified nucleotide (“C”, “A”, “G”, “U”)
2′Ome chemical modification=“m”
Position of the modified nucleotide relative to the reference sequence NR_046235.3 (NCBI, Mar. 10, 2017), nucleotide 1 corresponding to the first nucleotide of the human sequence encoding each rRNA of interest, either 28S (SEQ ID NO: 1), 18S (SEQ ID NO: 2) and 5.8S (SEQ ID NO: 3).

For example, “5.85_Um14” refers to 2′Ome on the nucleotide at position 14 of 5.8S rRNA corresponding to a U uridine.

This nomenclature is not valid for some sites because of an “ancestral” nomenclature kept for publication in order to allow a comparison with yeast in particular.

The 106 total sites of methylation in ribosomal RNAs are listed in Table 1 below, wherein the different columns have the following significance: “Figures” is the nomenclature used in the annexed figures, the legends of which are described here after, “NR_046235.Genomic_Position” is the position of the modified nucleotide relative to the reference sequence, “Official Nomenclature” is the one described above as the normalized method.

TABLE 1

| | | NR_046235.Geno- | Official |
|---|---|---|---|
| | FIGS. | mic_Position | Nomenclature |
| 1 | 28S_Am389 | 398 | 28S_Am398 |
| 2 | 28S_Am391 | 400 | 28S_Am400 |
| 3 | 28S_Gm1303 | 1316 | 28S_Gm1316 |
| 4 | 28S_Am1313 | 1326 | 28S_Am1326 |
| 5 | 28S_Cm1327 | 1340 | 28S_Cm1340 |
| 6 | 28S_Gm1509 | 1522 | 28S_Gm1522 |
| 7 | 28S_Am1511 | 1524 | 28S_Am1524 |
| 8 | 28S_Am1521 | 1534 | 28S_Am1534 |
| 9 | 28S_Gm1612 | 1625 | 28S_Gm1625 |
| 10 | 28S_Gm1747 | 1760 | 28S_Gm1760 |
| 11 | 28S_Am1858 | 1871 | 28S_Am1871 |
| 12 | 28S_Cm1868 | 1881 | 28S_Cm1881 |
| 13 | 28S_Cm2338 | 2351 | 28S_Cm2351 |
| 14 | 28S_Am2350 | 2363 | 28S_Am2363 |
| 15 | 28S_Gm2351 | 2364 | 28S_Gm2364 |
| 16 | 28S_Cm2352 | 2365 | 28S_Cm2365 |
| 17 | 28S_Am2388 | 2401 | 28S_Am2401 |
| 18 | 28S_Um2402 | 2415 | 28S_Um2415 |
| 19 | 28S_Cm2409 | 2422 | 28S_Cm2409 |
| 20 | 28S_Gm2411 | 2424 | 28S_Gm2424 |
| 21 | 28S_Am2774 | 2787 | 28S_Am2787 |
| 22 | 28S_Cm2791 | 2804 | 28S_Cm2804 |
| 23 | 28S_Am2802 | 2815 | 28S_Am2815 |
| 24 | 28S_Cm2811 | 2824 | 28S_Cm2824 |
| 25 | 28S_Um2824 | 2837 | 28S_Um2837 |
| 26 | 28S_Cm2848 | 2861 | 28S_Cm2861 |
| 27 | 28S_Gm2863 | 2876 | 28S_Gm2876 |
| 28 | 28S_Cm3680 | 3701 | 28S_Cm3701 |
| 29 | 28S_Am3697 | 3718 | 28S_Am3718 |
| 30 | 28S_Am3703 | 3724 | 28S_Am3724 |
| 31 | 28S_Gm3723 | 3744 | 28S_Gm3744 |
| 32 | 28S_Am3739 | 3760 | 28S_Am3760 |
| 33 | 28S_Am3764 | 3785 | 28S_Am3785 |
| 34 | 28S_Gm3771 | 3792 | 28S_Gm3792 |
| 35 | 28S_Cm3787 | 3808 | 28S_Cm3808 |
| 36 | 28S_Psi-Um3797 | 3818 | 28S_Psi-Um3818 |
| 37 | 28S_Am3804 | 3825 | 28S_Am3825 |
| 38 | 28S_Am3809 | 3830 | 28S_Am3830 |
| 39 | 28S_Cm3820 | 3841 | 28S_Cm3841 |
| 40 | 28S_Am3846 | 3867 | 28S_Am3867 |
| 41 | 28S_Cm3848 | 3869 | 28S_Cm3869 |
| 42 | 28S_Cm3866 | 3887 | 28S_Cm3887 |
| 43 | 28S_Gm3878 | 3899 | 28S_Gm3899 |
| 44 | 28S_Um3904 | 3925 | 28S_Um3925 |
| 45 | 28S_Gm3923 | 3944 | 28S_Gm3944 |
| 46 | 28S_Gm4020 | 4042 | 28S_Gm4042 |
| 47 | 28S_Cm4032 | 4054 | 28S_Cm4054 |
| 48 | 28S_Gm4166 | 4196 | 28S_Gm4196 |
| 49 | 28S_Um4197 | 4227 | 28S_Um4227 |
| 50 | 28S_Gm4198 | 4228 | 28S_Gm4228 |
| 51 | 28S_Um4276 | 4306 | 28S_Um4306 |
| 52 | 28S_Gm4340 | 4370 | 28S_Gm4370 |
| 53 | 28S_Gm4362 | 4392 | 28S_Gm4392 |
| 54 | 28S_Cm4426 | 4456 | 28S_Cm4456 |
| 55 | 28S_Gm4464 | 4494 | 28S_Gm4494 |
| 56 | 28S_Um4468 | 4498 | 28S_Um4498 |
| 57 | 28S_Gm4469 | 4499 | 28S_Gm4499 |
| 58 | 28S_Am4493 | 4523 | 28S_Am4523 |
| 59 | 28S_Cm4506 | 4536 | 28S_Cm4536 |
| 60 | 28S_Am4541 | 4571 | 28S_Am4571 |
| 61 | 28S_Am4560 | 4590 | 28S_Am4590 |
| 62 | 28S_Gm4588 | 4618 | 28S_Gm4618 |
| 63 | 28S_Um4590 | 4620 | 28S_Um4620 |
| 64 | 28S_Gm4593 | 4623 | 28S_Gm4623 |
| 65 | 28S_Gm4607 | 4637 | 28S_Gm4637 |
| 66 | 18S_Am27 | 27 | 18S_Am27 |
| 67 | 18S_Am99 | 99 | 18S_Am99 |
| 68 | 18S_Um116 | 116 | 18S_Um116 |
| 69 | 18S_Um121 | 121 | 18S_Um121 |
| 70 | 18S_Am159 | 159 | 18S_Am159 |
| 71 | 18S_Am166 | 166 | 18S_Am166 |
| 72 | 18S_Um172 | 172 | 18S_Um172 |
| 73 | 18S_Cm174 | 174 | 18S_Cm174 |
| 74 | 18S_Um428 | 428 | 18S_Um428 |
| 75 | 18S_Gm436 | 436 | 18S_Gm436 |
| 76 | 18S_Cm462 | 462 | 18S_Cm462 |
| 77 | 18S_Am468 | 468 | 18S_Am468 |
| 78 | 18S_Am484 | 484 | 18S_Am484 |
| 79 | 18S_Gm509 | 509 | 18S_Gm509 |
| 80 | 18S_Am512 | 512 | 18S_Am512 |
| 81 | 18S_Cm517 | 517 | 18S_Cm517 |
| 82 | 18S_Am576 | 576 | 18S_Am576 |
| 83 | 18S_Am590 | 590 | 18S_Am590 |
| 84 | 18S_Gm601 | 601 | 18S_Gm601 |
| 85 | 18S_Um627 | 627 | 18S_Um627 |
| 86 | 18S_Gm644 | 644 | 18S_Gm644 |
| 87 | 18S_Am668 | 668 | 18S_Am668 |
| 88 | 18S_Gm683 | 683 | 18S_Gm683 |
| 89 | 18S_Cm797 | 797 | 18S_Cm797 |
| 90 | 18S_Um799 | 799 | 18S_Um799 |
| 91 | 18S_Gm867 | 867 | 18S_Gm867 |
| 92 | 18S_Am1031 | 1031 | 18S_Am1031 |
| 93 | 18S_Cm1272 | 1272 | 18S_Cm1272 |
| 94 | 18S_Um1288 | 1288 | 18S_Um1288 |
| 95 | 18S_Psi-Um1326 | 1326 | 18S_Psi-Um1326 |
| 96 | 18S_Gm1328 | 1328 | 18S_Gm1328 |
| 97 | 18S_Am1383 | 1383 | 18S_Am1383 |
| 98 | 18S_Cm1391 | 1391 | 18S_Cm1391 |
| 99 | 18S_Um1442 | 1442 | 18S_Um1442 |
| 100 | 18S_Gm1447 | 1447 | 18S_Gm1447 |
| 101 | 18S_Gm1490 | 1490 | 18S_Gm1490 |
| 102 | 18S_Am1678 | 1678 | 18S_Am1678 |
| 103 | 18S_Cm1703 | 1703 | 18S_Cm1703 |
| 104 | 18S_Um1804 | 1804 | 18S_Um1804 |
| 105 | 5.8S_Um14 | 14 | 5.8S_Um14 |
| 106 | 5.8S_Gm75 | 75 | 5.8S_Gm75 |

As mentioned above, the methods of the present invention allow identification of rRNA 2′O-methylation-related signatures which carry clinical information, these signatures corresponding (i) either to a global 2′O-methylation signature based on all the 2′O-methylated rRNA sites containing both stable sites and variable sites, (ii) or a site-specific signature whose source corresponds to only some variable sites.

In the context of the present invention, levels of the 2′O-ribose methylation can be used in two different ways to provide relevant information:
(1) the 2′Ome whole profile integrating the information relating to all the sites: for a given sample, the levels of the 2′O-ribose methylation of all the sites are used and make it possible to establish a signature specific to the sample. This signature, including all the values of methylation level and thus all the combinatory of the methylation level of all the sites, allows samples to be compared with each other on the basis of their 2′Ome profile. Such analysis is herein called as “whole profile analysis”.
(2) The level of the 2′O-ribose methylation at a given site: for a given sample, the individual level of the 2′O-ribose methylation at a position of interest is used to compare samples with each other. In this case, it is the relative variation in the level of the 2′O-ribose methylation between samples at a position of interest that carries the information. Such analysis is herein called as “position-by- position analysis”.
The inventors have demonstrated that these two analysis of the 2′Ome values (i.e., values of all the sites=2′Ome profile of the individual or 1 value=2′Ome site of interest) can be used in the applications of the method of the present invention described here after, for diagnosis, for determining prognostic, for estimating benefit to a treatment.

Applying the method of the invention to several populations of patients, each being representative of one type of cancers, also allow to find a kind of “universal” signature, comprising the common variable sites which are not specific to a cancer type, by comparing the respective selections of potentially relevant markers identified by performing the method of the invention.

Therefore, according to an advantageous embodiment, the set of variable sites selected at step c) of the method of the invention contains from 5 to 50 2′O-ribose methylation positions among the 106 2′O-ribose methylation positions of rRNAs whatever the cancer the patients are suffering from, more preferably the set of variable sites selected at step c) contains from 5 to 10, from 5 to 15, from 5 to 20, from 5 to 25, from 5 to 30, from 5 to 35, from 5 to 40, from 5 to 45, from 5 to 50, from 10 to 15, from 10 to 20, from 10 to 25, from 10 to 30, from 10 to 35, from 10 to 40, from 10 to 45, from 10 to 50, from 15 to 20, from 15 to 25, from 15 to 30, from 15 to 35, from 15 to 40, from 15 to 45, from 15 to 50, from 20 to 25, from 20 to 30, from 20 to 35, from 20 to 40, from 20 to 45, from 20 to 50, from 25 to 30, from 25 to 35, from 25 to 40, from 25 to 45, from 25 to 50, from 30 to 35, from 30 to 40, from 30 to 45, from 30 to 50, from 35 to 40, from 35 to 45, from 35 to 50, from 40 to 45, from 40 to 50, from 45 to 50.

The set of 2′Ome positions selected by the method of the invention as potentially relevant markers in cancer prognosis and/or estimation of treatment benefit and/or therapy whatever the cancer type contains 5%, 10%, 15%, 20%, 25%, 30%, 35%, 40%, 45% or 50% of all the 2′Ome positions on rRNAs, preferably of the 106 positions as listed in Table 1.

Preferably, the ranges and proportions of variable positions as mentioned above apply to the set selected at step c) of the method of the invention and are referring to the number of variable sites which are in common between at least the following three type of cancers: breast cancers, B lymphoma and AML.

The method of the invention is relevant for any type of cancers.

However, the cancers for which it is advantageously relevant are selected among solid and hematologic cancers of adults and pediatric cancers.

In one preferred embodiment, the method of the invention is carried out on a representative population of patients suffering from solid cancers, more preferably from breast cancers, from glioma, more preferably from astrocytoma, glioblastoma or oligodendroglioma, from lymphoma, more preferably from B lymphoma, or from leukemia, more preferably acute myeloid leukemia (AML), and pediatric cancers, more preferably rhabdomyosarcoma and diffuse intrinsic pontine glioma (DIPG).

The most preferred representative population to which the method of the invention is applied is one of patients suffering from breast cancer or glioma.

Preferably, the set selected at step c) of the method according to the invention contains 11 positions among the 106 2′O-ribose methylation positions of rRNAs, and more preferably the 11 following positions in accordance with the nomenclature used in the annexed figures as explicated in Table 1:

### 18S_Um799; 28S_Am2774; 28S_Am4541; 28S_Cm1868; 28S_Cm2409; 28S_Gm1303; 28S_Gm3923; 28S_Gm4588; 28S_Gm4593; 28S_Gm4607; 28S_Um4590.

Said differently and according to official nomenclature, the above-preferred set contains the 11 2′O-ribose methylation positions:

### 18S_Um799; 28S_Am2787; 28S_Am4571; 28S_Cm1881; 28S_Cm2409; 28S_Gm1316; 28S_Gm3944; 28S_Gm4618; 28S_Gm4623; 28S_Gm4637; 28S_Um4620.

This set is particularly advantageous as being potentially relevant markers for diagnosis, prognosis and/or therapeutics in patients suffering from breast cancer, B lymphoma or AML.

Also preferably, the set selected at step c) of the method according to the invention contains the 13 positions among the 106 2′O-ribose methylation positions of rRNAs, and more preferably the 13 following positions in accordance with the nomenclature used in the annexed figures as explicated in Table 1:

### 18S_Am1031; 18S_Am590; 18S_Cm462; 28S_Am1858; 28S_Am2388; 28S_Am3703; 28S_Am3809; 28S_Am391; 28S_Cm4506; 28S_Gm2411; 28S_Gm4469; 28S_Um3904; 28S_Um4468.

Said differently and according to official nomenclature, the above-preferred set contains the 13 2′O-ribose methylation positions:

### 18S_Am1031; 18S_Am590; 18S_Cm462; 28S_Am1871; 28S_Am2401; 28S_Am3724; 28S_Am3830; 28S_Am400; 28S_Cm4536; 28S_Gm2424; 28S_Gm4499; 28S_Um3925; 28S_Um4498.

This set is particularly advantageous as being potentially relevant markers for diagnosis, prognosis and/or therapeutics in patients suffering from breast cancer or AML.

Still preferably, the set selected at step c) of the method according to the invention contains the 34 positions among the 106 2′O-ribose methylation positions of rRNAs, and more preferably the 34 following positions in accordance with the nomenclature used in the annexed figures as explicated in Table 1:

18S_Am27; 18S_Am468; 18S_Am484; 18S_Am512; 18S_Cm1272; 18S_Cm797; 18S_Gm1447; 18S_Gm436; 18S_Gm867; 18S_Um428; 18S_Um627; 18S_Um799; 28S_Am1313; 28S_Am2774; 28S_Am3846; 28S_Am4541; 28S_Cm1327; 28S_Cm1868; 28S_Cm2409; 28S_Cm3680; 28S_Gm1303; 28S_Gm2863; 28S_Gm3723; 28S_Gm3923; 28S_Gm4020; 28S_Gm4340; 28S_Gm4464; 28S_Gm4588; 28S_Gm4593; 28S_Gm4607; 28S_Psi-Um3797; 28S_Um2402; 28S_Um4590; 5.8S_Um14.

Said differently and according to official nomenclature, the set selected at step c) of the method according to the invention contains the 34 following positions:

18S_Am27; 18S_Am468; 18S_Am484; 18S_Am512; 18S_Cm1272; 18S_Cm797; 18S_Gm1447; 18S_Gm436; 18S_Gm867; 18S_Um428; 18S_Um627; 18S_Um799; 28S_Am1326; 28S_Am2787; 28S_Am3867; 28S_Am4571; 28S_Cm1340; 28S_Cm1881; 28S_Cm2409; 28S_Cm3701; 28S_Gm1316; 28S_Gm2876; 28S_Gm3744; 28S_Gm3944; 28S_Gm4042; 28S_Gm4370; 28S_Gm4494; 28S_Gm4618; 28S_Gm4623; 28S_Gm4637; 28S_Psi-Um3818; 28S_Um2415; 28S_Um4620; 5.8S_Um14.

This set is particularly advantageous as being potentially relevant markers for diagnosis, prognosis and/or therapeutics in patients suffering from breast cancer and B lymphoma.

According to a preferred embodiment, the method of the invention comprises measuring the 2′O-ribose methylation level of the 2′O-ribose methylation positions of rRNAs in biological samples from a representative population of patients suffering from breast cancer. In this preferred embodiment, the set of 2′O-ribose methylation positions selected at step c) contains from 40 to 50 2′O-methylation positions among the 106 2′O-ribose methylation positions of rRNAs, and more preferably the 46 2′O-ribose methylation positions in accordance with the nomenclature used in the annexed figures as explicated in Table 1:

18S_Am1678; 18S_Am27; 18S_Am468; 18S_Am484; 18S_Am512; 18S_Am576; 18S_Am668; 18S_Cm1272; 18S_Cm797; 18S_Gm1447; 18S_Gm436; 18S_Gm867; 18S_Um116; 18S_Um428; 18S_Um627; 18S_Um799; 28S_Am1313; 28S_Am2350; 28S_Am2774; 28S_Am2802; 28S_Am3739; 28S_Am3846; 28S_Am389; 28S_Am4541; 28S_Cm1327; 28S_Cm1868; 28S_Cm2352; 28S_Cm2409; 28S_Cm2848; 28S_Cm3680; 28S_Cm4032; 28S_Cm4426; 28S_Gm1303; 28S_Gm2863; 28S_Gm3723; 28S_Gm3923; 28S_Gm4020; 28S_Gm4340; 28S_Gm4464; 28S_Gm4588; 28S_Gm4593; 28S_Gm4607; 28S_Psi-Um3797; 28S_Um2402; 28S_Um4590; 5.8S_Um14.

Said differently and according to official nomenclature, the above-preferred set contains the 46 2′O-ribose methylation positions:

18S_Am1678; 18S_Am27; 18S_Am468; 18S_Am484; 18S_Am512; 18S_Am576; 18S_Am668; 18S_Cm1272; 18S_Cm797; 18S_Gm1447; 18S_Gm436; 18S_Gm867; 18S_Um116; 18S_Um428; 18S_Um627; 18S_Um799; 28S_Am1326; 28S_Am2363; 28S_Am2787; 28S_Am2815; 28S_Am3760; 28S_Am3867; 28S_Am398; 28S_Am4571; 28S_Cm1340; 28S_Cm1881; 28S_Cm2365; 28S_Cm2409; 28S_Cm2861; 28S_Cm3701; 28S_Cm4054; 28S_Cm4456; 28S_Gm1316; 28S_Gm2876; 28S_Gm3744; 28S_Gm3944; 28S_Gm4042; 28S_Gm4370; 28S_Gm4494; 28S_Gm4618; 28S_Gm4623; 28S_Gm4637; 28S_Psi-Um3818; 28S_Um2415; 28S_Um4620; 5.8S_Um14.

According to an also preferred embodiment, the method of the invention comprises measuring the 2′O-ribose methylation level of the 2′O-ribose methylation positions of rRNAs in biological samples from a representative population of patients suffering from glioma. In this preferred embodiment, the set of 2′O-ribose methylation positions selected at step c) contains from 30 to 40 2′O-methylation positions among the 106 2′O-ribose methylation positions of rRNAs, and more preferably the 32 2′O-ribose methylation positions in accordance with the nomenclature used in the annexed figures as explicated in Table 1:

18S_Am576; 18S_Cm1272; 18S_Cm174; 18S_Gm1447; 18S_Um116; 28S_Gm1747; 28S_Am2350; 28S_Am2388; 28S_Am3739; 28S_Am3764; 28S_Am3804; 28S_Am3846; 28S_Am391; 28S_Am4493; 28S_Am4541; 28S_Am4560; 28S_Cm1327; 28S_Cm2811; 28S_Cm2848; 28S_Cm4506; 28S_Gm2863; 28S_Gm3771; 28S_Gm3923; 28S_Gm4020; 28S_Gm4464; 28S_Gm4469; 28S_Gm4588; 28S_Gm4593; 28S_Gm4607; 28S_Um2402; 28S_Um2824; 28S_Um4590.

Said differently and according to official nomenclature, the above-preferred set contains the 32 2′O-ribose methylation positions:

18S_Am576; 18S_Cm1272; 18S_Cm174; 18S_Gm1447; 18S_Um116; 28S_Gm1760; 28S_Am2363; 28S_Am2401; 28S_Am3760; 28S_Am3785; 28S_Am3825; 28S_Am3867; 28S_Am400; 28S_Am4523; 28S_Am4571; 28S_Am4590; 28S_Cm1340; 28S_Cm2824; 28S_Cm2861; 28S_Cm4536; 28S_Gm2876; 28S_Gm3792; 28S_Gm3944; 28S_Gm4042; 28S_Gm4494; 28S_Gm4499; 28S_Gm4618; 28S_Gm4623; 28S_Gm4637; 28S_Um2415; 28S_Um2837; 28S_Um4620.

In this preferred embodiment with respect to a representative population of patients suffering from breast cancer and glioma, the most variable 2′O-ribose methylation position is 18S_Gm1447. In all the applications methods of the present invention described hereafter, the “representative population” is to be adapted to the dedicated respective applications. For example, for diagnosis purpose, the “representative population” is composed of a population of patients for which the cancer subtype is known. For prognostic purpose, the “representative population” is composed of a population of patients for which the prognostic is known. For estimating benefit to a treatment, the “representative population” is composed of a population of patients for which the benefit of the said treatment is known. In these cases, the “representative population” is thus corresponding to a “reference population”.

In a second aspect, the present invention relates to a method for determining the prognostic of a patient suffering from cancer irrespective of the treatment, comprising:
a) measuring the 2′O-ribose methylation level of the 2′O-ribose methylation positions of ribosomal RNAs (rRNAs) in a sample from a patient suffering from a cancer of whom the prognostic is to be determined, called “tested patient”,
b) comparing of the 2′O-ribose methylation level of the 2′O-ribose methylation positions of rRNAs measured with that of a representative population of patients suffering from the same cancer than the “tested patient” and for whom the clinical outcome is known,
c) determining the prognostic of the “tested patient” by identifying the one corresponding to the group of patients from the representative population which has the 2′O-ribose methylation level measures closer to that of the “tested patient”.

By “prognostic”, it is intended to mean the probable evolution of the disease which can be measured for example in terms of survival, disease progression, in particular determination of local or distant relapse, tumor grade and size, risk stratification, cancer subtypes. The identification of subtype of a cancer with particular outcome may be based on molecular consideration including genetic and/or genomic alterations, chromosomic alterations or particular gene expression, for example in hormone-dependent cancers in female or in IDH mutation status-dependent glioma.

Where survival is to be determined by the method of the invention, it may involve overall survival, disease-free survival, progression-free survival, relapse-free survival, both locally or at distance of the primary tumour site, hazard ratio or odd ratio.

By performing the method for determining the prognostic of a patient according to the invention, the prognostic may be either determined or more precisely defined. For example, although breast cancer patients carrying a small tumor size at diagnosis (<20 mm) usually exhibit a good outcome compared to patients carrying a large tumor size at diagnosis, molecular markers are needed to help to identify patients carrying a small tumour size however having an outcome as poor as patients carrying large tumour. Similarly, although glioma patient carrying a tumor of high grade at diagnosis (>stage III) usually exhibit the poorest outcome compared to all glioma patients, molecular markers are needed to help in identifying patients carrying a high-grade tumor however having a better outcome than other high grade glioma patients to adapt therapeutic strategy.

By performing the method for determining the prognostic of a patient according to the invention, where prognostic is in terms of tumor grade and size and/or cancer subtypes, said method may be integrated into the diagnostic stage of said patient and thus may be defined as a diagnostic method. Indeed, tumor grade and size and/or molecular cancer subtypes very often belong to the diagnostic of cancer in the patient. Therefore, as the meaning of prognosis may encompass cancer subtypes diagnosis, all details and embodiments herein regarding determination of the prognostic of a patient in the context of the present invention are also applying to cancer subtypes diagnosis.

In the method for determining the prognostic according to the invention, levels of the 2′O-ribose methylation can be used in two different ways to provide relevant information:
(1) the 2′Ome whole profile integrating the information relating to all the sites: for a given sample, the levels of the 2′O-ribose methylation of all the sites are used and make it possible to establish a signature specific to the sample. This signature, including all the values of methylation level and thus all the combinatory of the methylation level of all the sites, allows samples to be compared with each other on the basis of their 2′Ome profile. Such analysis is herein called as “whole profile analysis”.
(2) The level of the 2′O-ribose methylation at a given site: for a given sample, the individual level of the 2′O-ribose methylation at a position of interest is used to compare samples with each other.

In this case, it is the relative variation in the level of the 2′O-ribose methylation between samples at a position of interest that carries the information. Such analysis is herein called as “position-by-position analysis”.

The inventors have demonstrated that these two analysis of the 2′Ome values (ie, values of all the sites=2′Ome profile of the individual or 1 value=2′Ome site of interest) can be used as prognostic, and preferably for determining the patient survival or tumour grade, or as molecular cancer subtypes diagnostic.

Therefore, in a preferred embodiment of the method for determining prognostic, the comparison at step b) is carried out using a 2′O-ribose methylation position-by-position analysis or using a whole profile analysis of all the 2′O-ribose methylation positions.

As mentioned above for the method for identifying potentially relevant markers according to the invention, the “level of methylation” of the different methylation sites of rRNAs, is to be detected by any relevant technology which is known in the art, particularly by any method allowing to unambiguously determine whether the 2′O position of a ribose is methylated or not, and for example by any one of the above-mentioned methods.

In a preferred embodiment of the method for determining prognostic of the invention, the 2′O-ribose methylation level of the 2′O-ribose methylation positions is determined by a C-score calculated using RiboMeth-seq method.

More preferably, levels of the 2′O-ribose methylation is measured for the 106 sites as listed in Table 1. Thus, the two above-mentioned analysis of the 2′Ome values, which will be composed of 106 values as the 2′Ome profile of the individual or 1 value at a given 2′Ome site of interest, can be used as prognostic, and preferably for determining the patient survival or tumour grade or as molecular cancer subtypes diagnostic.

In another particularly preferred embodiment of the method for determining the prognostic according to the invention, the comparison at step b) is carried out using a 2′O-ribose methylation position-by-position analysis from 1 to 6 of variable positions, which are accurate markers of a particular type of cancers with prognosis, and preferably of 1, 2, 3, 4, 5 or 6 of variable positions are accurate markers of a particular type of cancers with prognosis.

In a particularly advantageous embodiment, the method of the invention is for determining the prognostic in a patient suffering from a breast cancer and comprises the comparison of the 2′O-methylation levels with a 2′O-methylation position-by-position analysis limited to at least one of the four 2′O-methylation positions 18S-Gm1447; 28S-Gm1303; 28S-Gm4588 and 18S-Am576, and preferably the four 2′O-methylation positions.

Said differently and according to official nomenclature, the method of the invention is for determining the prognostic in a patient suffering from a breast cancer and comprises the comparison of the 2′O-methylation levels with a 2′O-methylation position-by-position analysis limited to at least one of the four 2′O-methylation positions 18S-Gm1447; 28S_Gm1316; 28S_Gm4618 and 18S-Am576, and preferably the four 2′O-methylation positions.

In this embodiment, the method of the invention is particularly relevant for determining prognostic in a patient selected among survival and preferably overall survival, tumor grade, breast cancer subtype and preferably among which estrogen and progesterone statuses.

The method for determining the prognostic is particularly relevant for determining the risk stratification that is the designation of the patient's risk to evolve in such and such a way. In this embodiment, the method thus allows to determine the risk stratification resulting from the molecular subtype determination or anatomopathological characteristics of the tumour, including tumor grade and size.

For performing step b) of the method of the invention for determining the prognostic, relating to the comparison of the 2′O-ribose methylation level of the 2′O-ribose methylation positions of rRNAs with that of a representative population of patients suffering from the same cancer than the “tested patient” and for whom the clinical outcome is known, two options are available:
either the measures of the 2′O-ribose methylation levels in the representative population were previously available,
or, the measures of the 2′O-ribose methylation levels in the representative population is obtained as an additional step, carried out in parallel, to those measured at step a) of the method in a sample from the “tested patient”.

In a third aspect, the present invention relates to a method for estimating the benefit of a treatment in a patient suffering from cancer, comprising:
a) measuring the 2′O-ribose methylation level of the 2′O-ribose methylation positions of ribosomal RNAs (rRNAs) in a sample from a patient suffering from a cancer for whom the benefit of a specific treatment is to be determined, called “tested patient”,
b) comparing of the 2′O-ribose methylation level of the 2′O-ribose methylation positions of rRNAs measured with that of a representative population of patients suffering from the same cancer than the “tested patient” and for whom the benefit of one or more specific treatments is known,
c) determining the expected benefit of the specific treatment for the “tested patient” by selecting the one corresponding to the group of patients from the representative population which has the 2′O-ribose methylation level measures closer to that of the “tested patient”.

Benefit of a treatment is herein defined as an increase in tumour response, and/or duration and/or quality of treated patients' life which can be measured, in particular in terms of survival, including overall survival, disease-free survival, progression-free survival, relapse-free survival, both locally or at distance of the primary tumour site, hazard ratio or odd ratio, but also response rate, disease progression, in particular determination of local or distance relapse,

The estimation of the benefit of a treatment in the method according to the invention is intended to mean the definition or the assessment or the follow-up of the benefit of a treatment in a patient. At the final stage of cancer, the method for estimating the benefit of treatment in a patient may be strictly limited to consider the quality of the treated patients' life, since other measures linked to any therapeutic effect advantage is to be disregarded.

In a preferred embodiment, the method for estimating the benefit of a treatment in a patient is performed for assessing the benefit of a treatment in a patient who is in a treatment efficacy failure. By “ treatment efficacy failure ” is meant a patient who has been insufficiently responsive to at least one previous treatment, but also a patient who has been as a satisfactory response to at least one previous treatment but which has presented at least one adverse event of moderate to severe intensity during the previous treatment(s) requiring discontinuation of treatment. “By insufficient response to at least one previous treatment” is meant a patient who has not presented a positive therapeutic response to one or more previous treatment(s).

In another preferred embodiment, the method for estimating the benefit of a treatment in a patient is performed for assessing the benefit of a treatment in a patient who is not yet treated and for whom the most promising first intention treatment is to be determined. In this embodiment, the method may be integrated into the diagnostic stage of said patient and thus may be defined as a diagnostic method.

As used herein, the word “treatment” means any anti-cancer therapy.

The expression “one or more treatment” as used herein means “one or more among the available treatments”, and in particular “one or more among the available treatments traditionally used”. On that basis, it is clear that in case only one treatment is available for a type of cancer, the method when performed may be associated with the conclusion that the patient will not benefit of this unique available treatment and then shall not be treated therewith. In that particular case, alternatives to the conventional treatments may however be considered, for example as those which could be identified by the method for selecting one or more therapeutic drug(s) targeting ribosomes according to the invention, and as described here below.

As mentioned above for the method for determining prognostic, and it is a preferred embodiment of the method for estimating the benefit of a treatment in a patient suffering from cancer, the comparison at step b) is carried out using a 2′O-ribose methylation position-by-position analysis or using a whole profile analysis of all the 2′O-ribose methylation positions.

As also mentioned above, the “level of methylation” of the different methylation sites of rRNAs, is to be detected by any relevant technology which is known in the art, particularly by any method allowing to unambiguously determine whether the 2′O position of a ribose is methylated or not, and for example by any one of the above-mentioned methods.

In a preferred embodiment of the method for estimating the benefit of a treatment according to the invention, the 2′O-ribose methylation level of the 2′O-ribose methylation positions is determined by a C-score calculated using RiboMeth-seq method.

More preferably, levels of the 2′O-ribose methylation is measured for the 106 sites as listed in Table 1. Thus, the two above-mentioned analysis of the 2′Ome values, which will be composed of 106 values as the 2′Ome profile of the individual or 1 value at a given 2′Ome site of interest, can be used for estimating the benefit of a treatment in a patient according to the invention.

In another particularly preferred embodiment of the method for estimating the benefit of a treatment in a patient according to the invention, the comparison at step b) is carried out using a 2′O-ribose methylation position-by-position analysis from 1 to 6 of variable positions, which are accurate markers of a particular type of cancers with prognosis, and preferably of 1, 2, 3, 4, 5 or 6 of variable positions are accurate markers of a particular type of cancers with prognosis.

For performing step b) of the method of the invention for estimating the benefit of a treatment, relating to the comparison of the 2′O-ribose methylation level of the 2′O-ribose methylation positions of rRNAs with that of a population of patients suffering from the same cancer than the tested patient and for whom the benefit of this specific treatment is known, two options are available:
either the measures of the 2′O-ribose methylation levels in the representative population were previously available,
or, the measures of the 2′O-ribose methylation levels in the representative population is obtained as an additional step, carried out in parallel, to those measured at step a) of the method in a sample from the “tested patient”.

In view of the above, this method may also be defined as a method for treating a patient a suffering from cancer, comprising:
a) measuring the 2′O-ribose methylation level of the 2′O-ribose methylation positions of ribosomal RNAs (rRNAs) in a sample from a patient suffering from a cancer who needs to be treated,
b) comparing of the 2′O-ribose methylation level of the 2′O-ribose methylation positions of rRNAs measured with that of a representative population of patients suffering from the same cancer than the patient who needs to be treated; and for whom the benefit of several specific treatments is known,
c) selecting the specific treatment corresponding to one of the group of patients from the representative population which has the 2′O-ribose methylation level measures closer to that of the patient who needs to be treated,
d) administering the selected specific treatment to the patient who needs to be treated.

The term “specific treatment” as used herein is meaning a treatment useful in cancer therapy, adapted in view of the type of cancer.

In this method of treatment, the specific treatment selected at step c) is administered to the patient who needs to be treated and the other specific treatments corresponding to those of the respective representative populations which has the 2′O-ribose methylation level measures not closer to that of the patient who needs to be treated, are not administered to the patient who needs to be treated.

In a fourth aspect, the present invention is related to a method for selecting one or more therapeutic drug(s) targeting ribosomes, useful for treating cancers, comprising:
determining the target region(s) on the ribosome corresponding to ribosomal region(s) which comprise one or more 2′O-ribose methylation positions, the 2′O-ribose methylation level of which being known to be associated with cancer, and
identifying one or more ribosome-targeting drugs, including antibiotics, directed to said target region(s).

In this aspect of the invention, the approach for determining the therapeutic relevant variable sites is carried out using a 2′O-ribose methylation position-by-position analysis.

The inventors have indeed demonstrated that the analysis of 2′Ome at an individual level (site-by-site or position-by-position) is also of high interest for therapeutic targeting. Indeed, when knowing that one or more variable site(s) is associated with a diagnostic or prognostic trait or estimation of the benefit of the treatment or with tumoral-related phenotypic traits, it allows identifying a region of interest in the ribosome to specifically target the ribosome exhibiting these specific alterations.

In the method for selecting one or more therapeutic drug(s) according to the invention, the determination of the target region(s) on the ribosome is based on the 3D structure of the ribosome which is known in the art, and notably using information available via access to public database which correspond to human ribosome structure solved by cryo-EM (14-15).

This method is based on the following general approach:
Analysis of the 2′Ome of patients
Identification of variable site(s) predictive of a particular molecular cancer subtype/poor prognosis and/or no or poor benefit of an available treatment and/or tumoral phenotypic traits, which correspond to the 2′O-ribose methylation positions, the 2′O-ribose methylation level of which is known to be associated with cancer,
Location of this/these variable site(s) on the structure of the ribosome,
Determination of the distance between this/these variable site(s) and the location of pocket binding of ribosome-targeting drugs on the structure of the ribosome,
Identification of one or more ribosome-targeting drugs allowing the inhibition of ribosome activity and whose pocket binding within the ribosome shows the smallest and equal distance to 1 or more variable site(s) predictive of a particular molecular cancer subtype/poor prognosis and/or no or poor benefit of a treatment and/or tumoral phenotypic traits.

Therefore, the method for selecting one or more therapeutic drug(s) targeting ribosomes, useful for treating cancers, according to the present invention is preferably wherein
the target region(s) on the ribosome are determined by the following approach:
   Analysis of the 2′Ome of patients
   Identification of variable site(s) predictive of a particular molecular cancer subtype/poor prognosis and/or no or poor benefit of an available treatment and/or tumoral phenotypic traits, which correspond to the 2′O-ribose methylation positions, the 2′O-ribose methylation level of which is known to be associated with cancer,
   Location of this/these variable site(s) on the structure of the ribosome,
   Determination of the distance between this/these variable site(s) and the location of pocket binding of ribosome-targeting drugs on the structure of the ribosome; and
the one or more ribosome-targeting drugs are identified by identifying the one or those allowing the inhibition of ribosome activity and whose pocket binding within the ribosome shows the smallest and equal distance to 1 or more variable site(s) predictive of a particular molecular cancer subtype/poor prognosis and/or no or poor benefit of a treatment and/or tumoral phenotypic traits.

The expression “no benefit of a treatment” is intended to mean a lack of response to an available treatment.

The expression “tumoral phenotypic traits” is intended to mean a 2′Omethylation site having been shown to promote tumor initiation or progression in in vitro, in cellulo or in vivo cancer models.

In another particularly preferred embodiment of this method, the analysis of the rRNA 2′Ome for identifying the therapeutic relevant variable site(s) is carried out using a 2′O-ribose methylation position-by-position analysis from 1 to 6 of variable positions, which are accurate markers of a particular type of cancers with prognosis, and preferably of 1, 2, 3, 4, 5 or 6 of variable positions are accurate markers of a particular type of cancers with prognosis.

As used herein the expression “known to be associated with cancer” means known to be predictive of a diagnostic or prognostic trait, such as poor prognosis and/or no or poor benefit of treatment and/or to have been shown to promote phenotypic traits related to cancer initiation and progression, for example in experimental models.

The analysis of the 2′Ome for identifying the therapeutic relevant variable site(s) is preferably performed by the method for determining prognostic and/or for estimating the benefit of a treatment in a patient suffering from cancer as described above in accordance with the present invention. By performing the analyses detailed above, which are used in the different aspects of the present invention, 2′O-ribose methylation position(s) the methylation level of which is predictive of a particular molecular cancer subtype/poor prognosis and/or no or poor benefit of a treatment may be determined and thus the corresponding target region(s) on the ribosome may be identified depending on the location of these predictive sites.
As used herein, the term “ribosome-targeting drugs” is referring to small molecule that binds to the ribosomes and impairs, or totally inhibits or modulate or modify its translational activities.

Identification of one or more ribosome-targeting drugs directed to the above-mentioned target region(s) in the ribosome is carried out by selecting the one or those whose target environment allows the impairment, or total inhibition or modulation or modification of ribosome activity and contains at least one of the therapeutic relevant variant site(s). This may notably be carried out using information available via access to public database. The target environment is constituted by the specific nucleotide(s) and/or amino acid(s) including at least one of the variable sites located in the target region.

Preferably, further to identifying one or more ribosome-targeting drugs directed to the above-mentioned target region(s) in the ribosome, selection of the most appropriate ribosome-targeting drugs useful for treating cancer is carried out among those already authorized on the market or susceptible to have the required characteristics for medical use.

In a preferred embodiment, identification of one or more ribosome-targeting drugs useful for treating cancers is carried out among the antibiotics or antibiotics families, and more preferably, among the aminoglycosides, tetracyclines, macrolides, chloramphenicol, lincosamide, linezolid and streptogramines.

As mentioned above, the “level of methylation” of the different methylation sites of rRNAs, is to be detected by any relevant technology which is known in the art, particularly by any method allowing to unambiguously determine whether the 2′O position of a ribose is methylated or not, and for example by any one of the above-mentioned methods.

In a preferred embodiment of the method for selecting one or more therapeutic drug(s) targeting ribosomes according to the invention, the 2′O-ribose methylation level of the 2′O-ribose methylation positions is determined by a C-score calculated using RiboMeth-seq method.

More preferably, levels of the 2′O-ribose methylation is measured for the 106 sites as listed in Table 1. Thus, the two above-mentioned analysis of the 2′Ome values, which will be composed of 106 values as the 2′Ome profile of the individual or 1 value at a given 2′Ome site of interest, can be used in this method for selecting one or more therapeutic drug(s) targeting ribosomes.

A preferred embodiment of the method for selecting one or more therapeutic drug(s) targeting ribosomes useful for treating cancers according to the invention, is based on the following more particular approach:
Analysis of the 2′Ome of patients, more preferably by RiboMeth-seq technology,
Identification of variable site(s) predictive of a particular molecular cancer subtype/poor prognosis and/or no or poor benefit of treatment and/or tumoral phenotypic traits, which correspond to the 2′O-ribose methylation positions, the 2′O-ribose methylation level of which is known to be associated with cancer,
Location of this/these variable sites on the structure of the ribosome,
Determination of the distance between this/these variable site(s) and the location of pocket binding of antibiotics on the structure of the ribosome,
Identification of antibiotics whose pocket allowing the impairment, or total inhibition or modulation or modification of ribosome activity and whose pocket binding within the ribosome shows the smallest and equal distance to 1 or more variant site(s) predictive of a particular molecular cancer subtype/poor prognosis and/or no or poor benefit of treatment.

In the method for selecting one or more therapeutic drug(s) targeting ribosomes useful for treating cancers according to the invention, two methodologies may be used:
performing the method for a representative population of patients suffering from a cancer to identify the predictive variable positions and thus the associated target region(s) in the ribosome,
performing the method by comparison between a “healthy” population consisting in patients not suffering from a cancer and a pathological representative population consisting in patients suffering from a cancer, to identify the variable positions which are specific of the pathological representative population, and thus identifying the associated target region(s) in the ribosome. In this embodiment, where the comparison is to be performed with a population of patients who do not suffer from a cancer, the “healthy” population is in fact corresponding to a “control population”. This is why in a preferred embodiment of the method for selecting one or more therapeutic drug(s) targeting ribosomes useful for treating cancers according to the invention the 2′O-ribose methylation positions associated with cancer are identified by:
a) measuring the 2′O-ribose methylation level of the 2′O-ribose methylation positions of ribosomal RNAs (rRNAs) in biological samples from a representative population of patients suffering from a cancer,
b) assessing the individual methylation status for each 2′O-ribose methylation positions by determining the variability of the 2′O-ribose methylation level thus measured for each 2′O-ribose methylation positions between each sample of patients from the representative population,
c) selecting the set of 2′O-ribose methylation positions for which the individual methylation status is a “variable status”. Preferably, assessment of the methylation status is carried out according to steps b1) and b2) as specified above.

Also, in another preferred embodiment of the method for selecting one or more therapeutic drug(s) targeting ribosomes useful for treating cancers according to the invention the 2′O-ribose methylation positions associated with cancer are identified by:
a) measuring the 2′O-ribose methylation level of the 2′O-ribose methylation positions of ribosomal RNAs (rRNAs) in biological samples from a representative population of patients suffering from a cancer and in biological samples from a population of patients not suffering from a cancer (control population),
b) assessing the individual methylation status for each 2′O-ribose methylation positions by determining the variability of the 2′O-ribose methylation level thus measured for each 2′O-ribose methylation positions between each sample of patients from the two populations (the representative population of patients suffering from a cancer and the control population of patients not suffering from a cancer),
c) selecting the set of 2′O-ribose methylation positions for which the individual methylation status is a “variable status” in the representative population of patients suffering from a cancer and not in the control population. Preferably, assessment of the methylation status is carried out according to steps b1) and b2) as specified above.

Stating differently, this step c) is selecting the set of 2′O-ribose methylation positions for which the individual methylation status is a “variable status” in the representative population of patients suffering from a cancer and not in population of patients not suffering from a cancer.

Once the most promising therapeutic drug targeting ribosomes useful for treating cancers have been selected by the method according to the invention, the ribosome-based drug may be used alone or in combination to a conventional treatment with the aim of improving global treatment efficiency. In a specific embodiment of the present method, it may lead to the selection of an antibiotic, preferably one selected among the above-mentioned antibiotics families, which may be used alone or in combination with a conventional treatment.

In all the methods of the invention, all the preferred embodiments mentioned for the method to identify potentially relevant markers with respect to the analysis approach involving determination of methylation level, methylation status, variable status also apply to all the other methods of the invention based thereon in order to determine particular molecular cancer subtype, prognosis, benefit of treatment and selection of therapeutic drug(s) targeting ribosomes or any other relevant application in clinical follow-up of patients.

In the context of the present invention, it is also described a kit comprising molecules able to specifically recognize the modified regions on the rRNAs containing the relevant markers which are accurately associated with a clinical significance in cancer. Any kind of molecules able to specifically detect the modified regions containing the 2′Ome site of interest may be contained in the kit of the invention, for example primers, probes or antibodies or fragment thereof which specifically detect at least one of the 2′Ome site(s) which is(are) accurately associated with a clinical significance in cancer.

In one preferred embodiment, the kit of the invention comprises molecules able to specifically recognize or detect at least one of the four 2′O-methylation positions 18S-Gm1447; 28S-Gm1303; 28S-Gm4588 and 18S-Am576 which are predictive in all the application methods described above, diagnostic, prognostic, benefit to treatment and selection of therapeutic drug(s) targeting ribosomes, for the patient is suffering from a breast cancer. Preferably, the kit comprises molecules able to specifically detect two, three or the four of these predictive variable sites.

As used herein, the expression “primers specifically targeting” one or more variable sites, means couple of primers with various sequences and able to specifically generate an amplicon encompassing the part of the nucleotide rRNA sequence containing said predictive variable sites.

As used herein, the term “primers” designates nucleic acid molecules that can specifically hybridize or anneal to 5′ or 3′ regions of the relevant target region on rRNAs. In general, they are from about 18 to 22 nucleotides in length and anneal at both extremities of a region containing about 60 to 120 nucleotides in length. As they have to be used by pairs, they are often referred to as “primers pair” or “primers set”.

As used herein, the term “probes” designates molecules that are capable of specifically hybridizing the rRNA region of interest.

In a preferred embodiment, the probes of the invention comprise at least 20, consecutive nucleotides which are complementary of the 2′O-methylation positions 18S-Gm1447; 28S-Gm1303; 28S-Gm4588 and 18S-Am576. In a more preferred embodiment, the molecules which can be used as a probe according to the present invention have a total minimum size of 19 nucleotides. In an even more preferred embodiment, these molecules comprise between 18 and 20 nucleotides (in total).

Among the technics which may be used for implementation of the kit according to the invention, it may be cited those with a PCR-based approaches for quantifying RT products (eg, RT-PCR, RT-qPCR or ddPCR), and which relies on the inhibition of reverse transcription reaction by 2′O-methylation at low dNTP concentration and on the detection of total rRNA as an internal reference, by reverse transcription at high dNTP concentration. For example, implementation of the kit of the invention may be as described in Belin et al, Plos One 2009 (10), in particular on the basis of FIG. 4, especially FIG. 4A.

The one skilled in the art is fully aware and know the appropriate conditions and appropriate reagents, so that primers or probes permit the amplification or hybridization of the rRNAs comprising the predictive variable sites of interest. The person skilled in the art has also the knowledge for generating antibodies specifically directed against the modified region(s) containing the 2′Ome site of interest, which may be comprised in the kit of the invention.

As used herein, the term “kit” refers to any system for delivering materials. In the context of the invention, it includes systems that allow the storage, transport, or delivery of reaction reagents (e.g., oligonucleotides, enzymes, and/or positive and negative controls from one site to another, etc. in the appropriate containers) and/or supporting materials (e.g., buffers, written instructions for performing the assay etc.). For example, kits include one or more enclosures (e.g., boxes) containing the relevant reaction reagents and/or supporting materials. The present kit can also include one or more reagents, buffers, hybridization media, nucleic acids, primers, nucleotides, probes, molecular weight markers, enzymes, solid supports such as beads and the like, databases, computer programs for analyzing the raw data and/or disposable laboratory equipment, such as multi-well plates, in order to readily facilitate implementation of the present methods. Enzymes that can be included in the present kits include nucleotide polymerases and the like. Molecules able to specifically detect stable site(s) or a synthetic RNA containing or not the 2′Ome can be used as controls in the kit of the invention.

Preferably, the kit or system of the invention useful for all the application methods described above (diagnostic, prognostic, benefit to treatment and selection of therapeutic drug(s) targeting ribosomes), for a patient suffering from a breast cancer, does not contain other molecules which specifically target, recognize or detect variable site(s) other than the four above-mentioned positions.

In addition, it is also described uses of this kit for the different applications defined in the methods described above, for molecular cancer subtypes diagnosis, for determining prognostic, for estimating benefit to a treatment and for selecting therapeutic drug(s) targeting ribosomes, in a patient suffering from a breast cancer. All the preferred embodiments described for these different application methods also apply to the use of the kit, as far as the patient is suffering from a breast cancer. Such uses are ‘“in vitro” or “ex vivo” performed.

Such kits and uses thereof represent additional aspects of the present invention.

### FIGURE LEGENDS

FIG. 1 represents reproducibility and robustness of RiboMeth-seq technology when sequencing human biological samples. The r2 correlation coefficient for the three rRNAs between technical duplicate of the 20 human breast tumours was shown as a cumulative level. More than 50%, 85% and 65% of technical duplicates showed a r2>0.8 for 5.8S, 18S and 28S rRNA respectively, the smallest correlation being observed for 5.8S rRNA, probably due to its small size compared to 18S and 28S (157 bp vs 1875 or 5035 bp, respectively).

FIG. 2 represents stability and variability of rRNA 2′O-methylation levels between breast tumours. Levels of rRNA 2′O-methylation (i.e., C-score) were determined at the 106 rRNA 2′O-methylated sites using RiboMeth-seq in a series of 195 primary human breast tumours. (a) Data are presented as a hierarchical clustering where the C-score of each position (column) for each tumour (row) is presented (shaded of grey scale). Dendrograms represent relationships of similarity between breast tumours on the basis of their rRNA 2′O-methylation profiles (left panel) that identify 4 groups of breast tumour samples (right panel, G1 to G4), or in the rRNA 2′O-methylation level at a given site between tumours (top panel). (b) is a table listing each RNA 2′OMe sites of (a) above from left to right X axis.

FIG. 3 represents biological relevance of variable rRNA 2′O-methylation levels in breast cancer. (a) C-score variation among the 195 human primary breast tumours at each of the 106 rRNA 2′O-methylated sites was ranked by increasing interquartile range (IQR). Based on IQR divergence, two classes of rRNA 2′O-methylated sites were defined: sites with the most stable C-scores and sites with the most variable C-scores. (b) is a table listing each RNA 2′OMe sites of (a) above from left to right X axis.

FIG. 4 represents an example of variability in rRNA 2′O-methylation levels at the rRNA 2′O-methylated site 18S-Gm1447 that show the highest variability in breast cancer. The plot presents the C-score (y-axis) for each of the 195 human primary breast tumours (x-axis) at this rRNA 2′O-methylated site. The dotted lines indicate the mean±2 standard deviations encompassing 95% of the primary breast tumours.

FIG. 5 represents variability of rRNA 2′O-methylation level between the 195 human primary breast tumours. Two classes of rRNA 2′O-methylated sites are defined based on the variability of their level among the patients based on the C-score inter-patient variability measured by interquartile range (IQR). The plot represents the IQR of C-scores calculated using the 195 human primary breast tumours at each of the 106 rRNA 2′O-methylated sites ranked by increasing IQR. The cut-off site is the site from which the IQR values no longer lie on this straight line, i.e. beyond which a straight line should change its slope to pass through them. This allowed the determination of two classes of rRNA 2′O-methylated sites: the ones with the most stable C-score; and the ones with the most variable C-score. Overall, these data indicate that 60% of the rRNA 2′O-methylated sites correspond to chemically modified nucleotides with conserved rRNA 2′O-methylation levels in humans, while 40% of these sites can be either 2′O-methylated or not.

FIG. 6 represents the comparison of the list of variable sites identified in tumor samples issued from patients suffering from three distinct cancer types: breast cancer, acute myeloid leukemia (AML) or splenic marginal zone lymphoma (SMZL) corresponding to B-cell lymphoma.

FIG. 7 represents biological and clinical relevance of variable rRNA 2′O-methylation sites in breast cancer. Summary of the four variable rRNA sites the 2′O-methylation level of which were significantly different between breast cancer subtypes, hormonal and HER2 receptor, and tumour grade. ER: oestrogen receptor; PR: progesterone receptor; Luminal: ER+ PR+/− HER2−; HER2+: ER− PR− HER2+; TNBC: ER− PR− HER2−; G: grade.

FIG. 8 represents association of rRNA 2′O-methylation level at site 18S-Gm1447 with breast cancer patients' outcome. Kaplan-Meier curve suggested the patients carrying breast tumours characterized by a low level of 2′O-methylation at site 18S-Gm1447 display the poorest overall survival.

FIG. 9 represents association of rRNA 2′O-methylation level at site 18S-Gm1447 with breast cancer progression. Kaplan-Meier curve suggested the patients carrying breast tumours characterized by a low level of 2′O-methylation at site 18S-Gm1447 display the poorest progression-free survival.

FIG. 10 represents association of rRNA 2′O-methylation profiles with breast cancer patients' outcome. Kaplan-Meier curve suggested the patients carrying breast tumours characterized by a G2 rRNA 2′O-methylation profile display the poorest overall survival.

FIG. 11 represents association of rRNA 2′O-methylation profiles with intrinsic breast cancer subtype. Significant differences in repartition among the 4 groups were observed regarding breast cancer subtype. In particular, the G2 group displaying closed rRNA 2′Ome profiles shows low frequency of HER2+ breast cancer subtype compared to others. Luminal: ER+ PR+/− HER2−; HER2+: ER− PR− HER2+; TNBC: ER− PR− HER2−.

FIG. 12 represents association of rRNA 2′O-methylation profiles with tumor grade. Significant differences in repartition among the 4 methylation groups were observed regarding tumour grade. In particular, the G1 group displaying closed rRNA 2′Ome profiles is devoid of grade 1 breast tumour.

FIG. 13 represents breast cancer patients' outcome depending of the statuses of their tumors regarding both size and 2′Ome profiles at diagnosis. Kaplan-Meier curve suggested the patients carrying small breast tumours characterized by a G2 rRNA 2′O-methylation profile display an overall survival as poor as patients carrying large breast tumours, the tumour of small size being usually associated with a good prognosis compared to the tumour of large size.

FIG. 14 represents association of rRNA 2′O-methylation profiles with survival of breast cancer patients treated with surgery and adjuvant radiotherapy/hormonotherapy. Kaplan-Meier curves show that patients carrying breast tumors characterized by closed rRNA 2′O-methylation profile display different outcomes (P=0.0665). In particular, compared to the global population treated with surgery+radiotherapy/hormonotherapy (black line), patients carrying tumors with G2 rRNA 2′O-methylation profile display a better overall survival than patients carrying tumors with G3 one.

FIG. 15 represents clustering of glioma tumors based on their rRNA 2′O-methylation profiles. A cohort of 46 brain samples was analyzed that were issued from 6 healthy donors (x in a square), 13 oligodendroglioma (square), 13 astrocytoma (circle) and 14 glioblastoma (triangle). Two internal controls were used to assess technical issued (X, internal control RNA1 and RNA2). Using this cohort, the Principal Component Analysis (PCA) method identified two groups of glioma tumors displaying similar rRNA 2′O-methylation profiles, one of them being composed of all the 14 glioblastoma (black open circle).

FIG. 16 represents association of rRNA 2′O-methylation profiles with overall and progression-free survival of glioma patients and of mitosis of glioma tumors. Correlation analyses between distinct rRNA 2′O-methylation profiles separated by dimensions of the PCA method (FIG. 15) and clinical data are given as correlation r2 coefficient and the associated p-value. Clustering of glioma tumors on two groups based on their similarity in rRNA 2′O-methylation profiles identified by dimension 2 is significantly associated with overall survival, progression-free survival and mitosis, all these characteristics being gold standard criteria to identified aggressive glioma tumors.

FIG. 17 represents identification of variable rRNA 2′Ome sites in mesenchymal cells compared to epithelial cells. rRNA 2′Ome level at the 106 positions in the epithelial hMEC cell line and in the mesenchymal hMEC-ZEB1 one has been analysed using RiboMeth-seq (n=5). A significant variation in rRNA 2′Ome level was observed for two sites (P-value <0.05): 28S-Um2402 and 28S-Gm4588, the 2′Ome levels of which is decreased and increased in mesenchymal cells compared to epithelial ones, respectively.

FIG. 18 represents identification of antibiotic binding regions encompassing variable rRNA 2′Ome sites in mesenchymal cells. The 2′Ome sites (spheroid dots) and binding pockets of well-known eukaryote-specific antibiotics (dotted circles) were located on the available human structure of the ribosome resolved by Cryo-EM (PDB, AUG0). The 28S-Um2402 and 28S-Gm4588 positions, the 2′Ome level of which varies between mesenchymal and epithelial cells, are in the vicinity of the binding pocket of the anisomycin antibiotic.

FIG. 19 represents increased sensibility of mesenchymal cells to antibiotic treatment compared to epithelial cells. Cell viability of epithelial and mesenchymal cells was monitored using real-time monitoring system in response to increasing concentration of anisomycin. The hMEC-ZEB1 mesenchymal cells are more sensitive to anisomycin treatment that the hMEC epithelial cells.

### EXAMPLES

### Example 1—Identification of Two Classes of rRNA 2′O-methylation Sites in Human Samples of Patients Suffering From Breast Cancer (Solid Tumours)

### Materials and Methods

Human samples. A series of 195 female primary breast tumours were collected from 1997 to 2011 and maintained by the Tayside Tissue Bank (TTB, Dundee, Scotland, UK) under ethical approval (REC Reference 07/S1402/90) (19). This retrospective series of primary breast tumours is composed of both non-invasive (45%) and invasive (55%) lymph node tumours. Total RNA was extracted from frozen tissues by TTB services as already described (19). This series exhibited expected clinical characteristics, in particular regarding association of overall survival with tumour size (P<0.0001***), lymph node invasion (P=0.0079″), grade (P=0.0151*) and breast cancer subtypes (P=0.0569). A human RNA reference sample (i.e., RNA reference) was used as a calibrated source of rRNA (Human XpressRef Universal Total RNA, Qiagen) prepared from 20 different human adult and foetal normal major organs.

RiboMeth-seq. Levels of rRNA 2′O-methylation at the 106 rRNA 2′O-methylated sites were determined by RiboMeth-seq (7-8, 13). Presence of 2′O-methylation protects the phosphodiester bond located at the 3′ of the 2′O-methylated nucleotide from alkaline hydrolysis. Thus, the presence of 2′O-methylation at the given nucleotide n induces under-representation of RNA fragments starting at the nucleotide n+1 and ending at position n allowing to calculate a 2′O-methylation level at the corresponding nucleotide position (or C-score) varying from 0 to 1 (8): a C-score of 0 meaning that all the rRNA molecules are not 2′O-methylated at the given site, a C-score of 1 indicating that all the rRNA are fully 2′O-methylated at the given site, and a 0 <C-score <1 meaning that the sample displays a mix of 2′O-methylated and un-2′O-methylated rRNA molecules at the given site. RiboMeth-seq was performed using the Illumina sequencing 25 technology and raw data were processed as previously described (7, 13). The median number of total reads reaches 7.2 millions after trimming, these reads being aligned on the 7.2 kb-long rRNA sequences, that corresponds to the optimal sequencing depth (7, 13).

Analysis of the breast cancer series. Among an initial series of 214 primary breast tumours, RiboMeth-seq data of 195 primary breast tumour samples passed the QC criteria (representing 91% of the initial series) and were thus retained for the downstream analyses. Unsupervised data analysis was performed (hierarchical clustering and principal component analysis) using the C-scores at the 106 individual rRNA 2′O-methylated sites of the 195 samples. rRNA 2′O-methylation profiles were shown as either box-and-whiskers plots, line charts or barcharts. Classification of the rRNA 2′O-methylation sites into “stable” and “variable” classes was done empirically based on the variability of the sites (in terms of interquartile range, IQR), the cut-off site being the site from which the IQR values no longer lie on this straight line. 2′O-methylated sites having IQR values below the threshold were termed “stable”, while sites having IQR values above were designated as “variable”. Statistical analyses and graphical representations were performed using either R v3.6.3 or GraphPad Prism v7.0a software (GraphPad Software, Inc).

rRNA 2′O-methylation evolution and mapping. The rRNA 2′O-methylated sites were mapped on the structure of the HeLa cancer cell human ribosome determined by cryo-EM (14-16) and images were drawn using the PyMol software. Observations were based on previous reported 3D molecular docking analysis of tRNAs or ribosome-associated factors, as already discussed in (ref 14-16).

### Results

### Optimization of RiboMeth-seq Technology for Human Samples

To profile rRNA 2′O-methylation in human primary breast tumours, we used the RiboMeth-seq technology. The measurement of 2′O-methylation level by RiboMeth-seq technology relies on a partial alkaline hydrolysis of the rRNA phosphodiester bonds, which become refractory to hydrolysis when adjacent riboses are methylated in position 2′ (7, 8, 13). RiboMeth-seq processing yields a score (i.e., C-score) at each of the 106 rRNA 2′O-methylated positions, reflecting the level of 2′O-methylation.

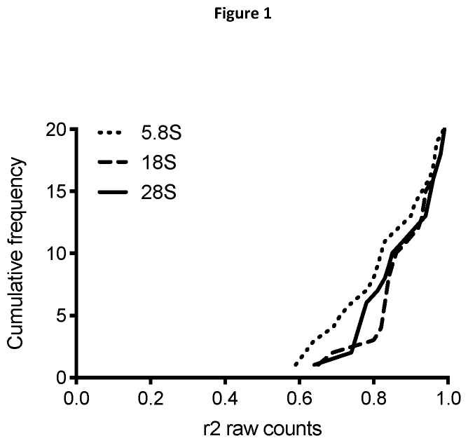

Since rRNA 2′O-methylation profiling has never been performed on large series of human samples, we developed RiboMeth-seq-dedicated extensive quality controls based on numerous metrics. Using human RNA reference samples composed of a mix of 20 different human adult and foetal normal major organs and a test set of 20 primary breast tumour samples, we show a strong correlation in the C-scores among the technical replicates (FIG. 1). Overall, our data demonstrate that the high-throughput RiboMeth-seq technology is a reproducible and robust technology even when sequencing delicate biological material such as frozen tumour biopsies.

Identification of Two Classes of rRNA 2′O-methylation Sites

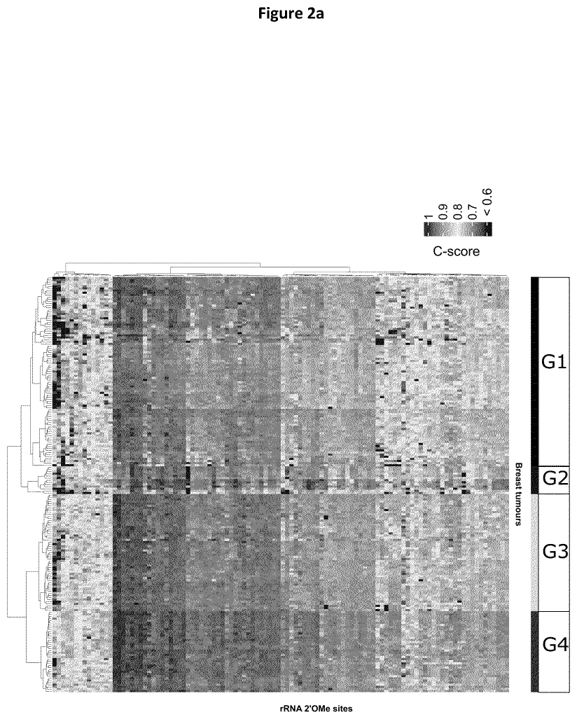

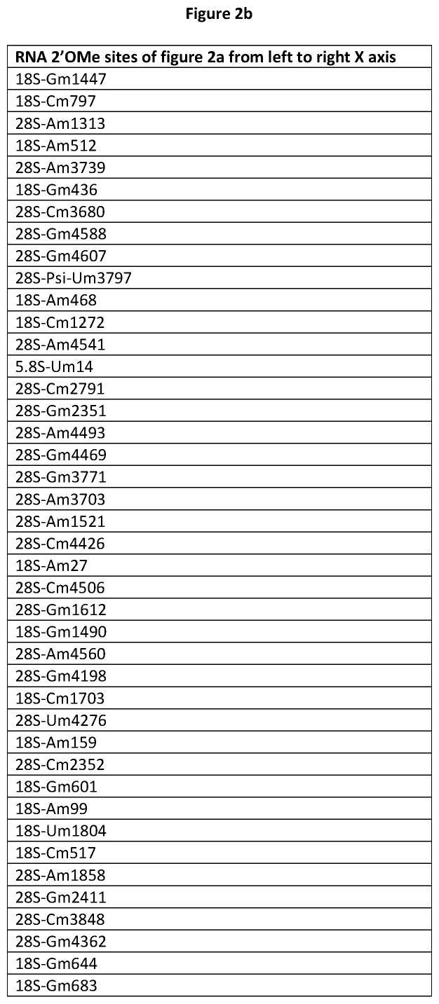

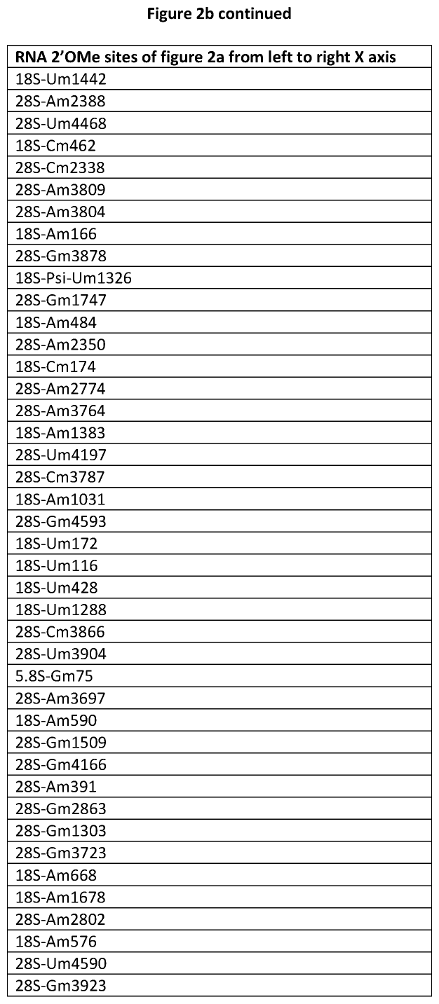

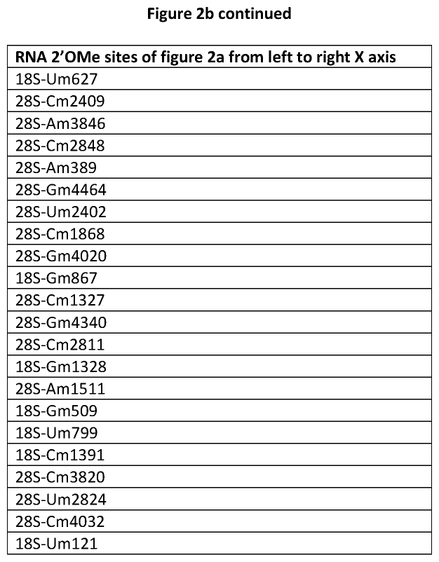

We then profiled rRNA 2′O-methylation in a series of 195 primary breast cancers composed of a mix of invasive and non-invasive tumours using RiboMeth-seq (12). Unsupervised statistical analysis revealed variability in rRNA 2′O-methylation levels (FIG. 2). It first showed that, for a given tumour, the 2′O-methylation level varies between 0 and 0.98 among the 106 rRNA 2′O-methylation sites. This demonstrates that in a single human tumour sample, all rRNA molecules are not fully and equally 2′O-methylated at the 106 sites.

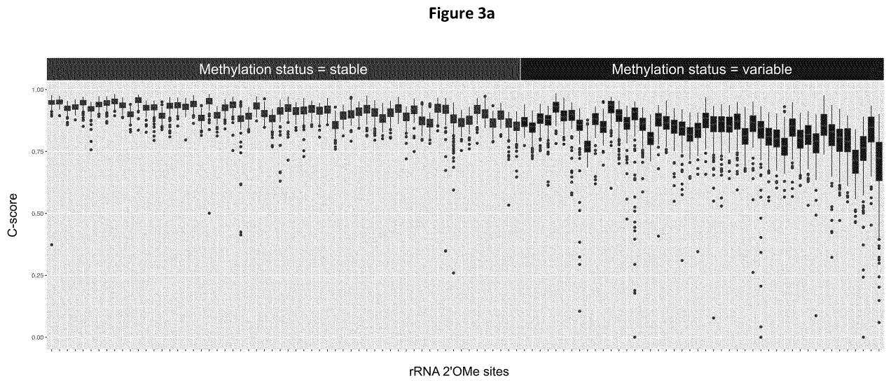

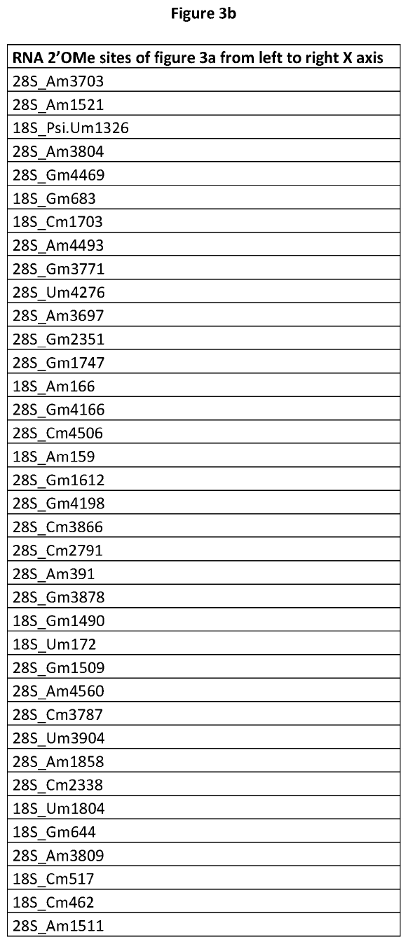

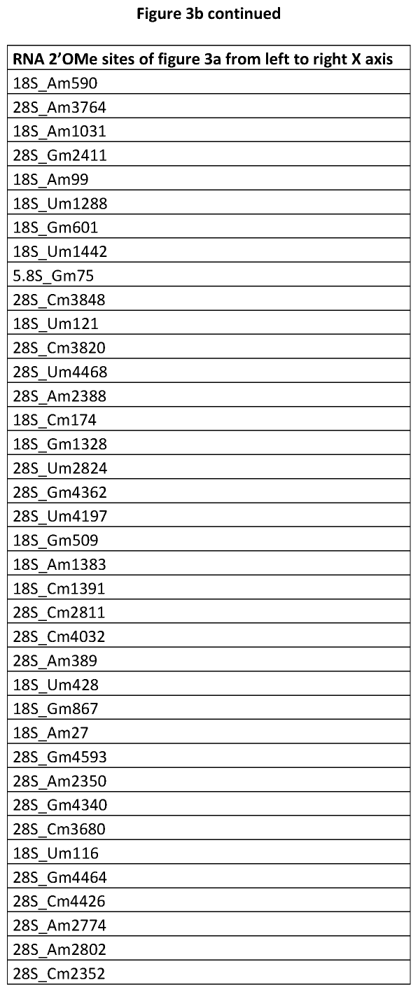

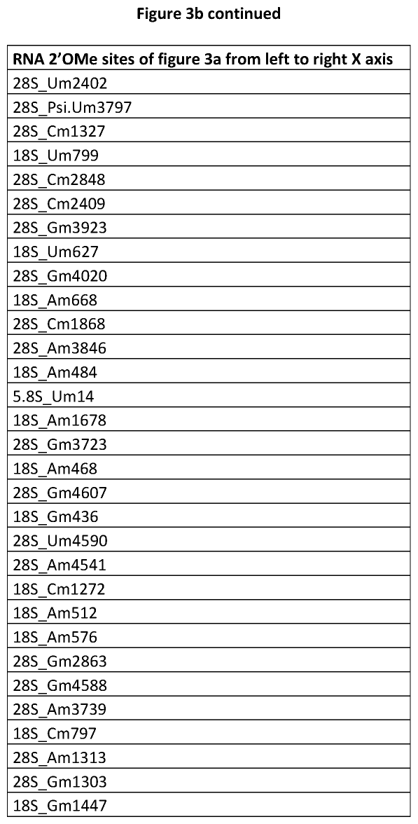

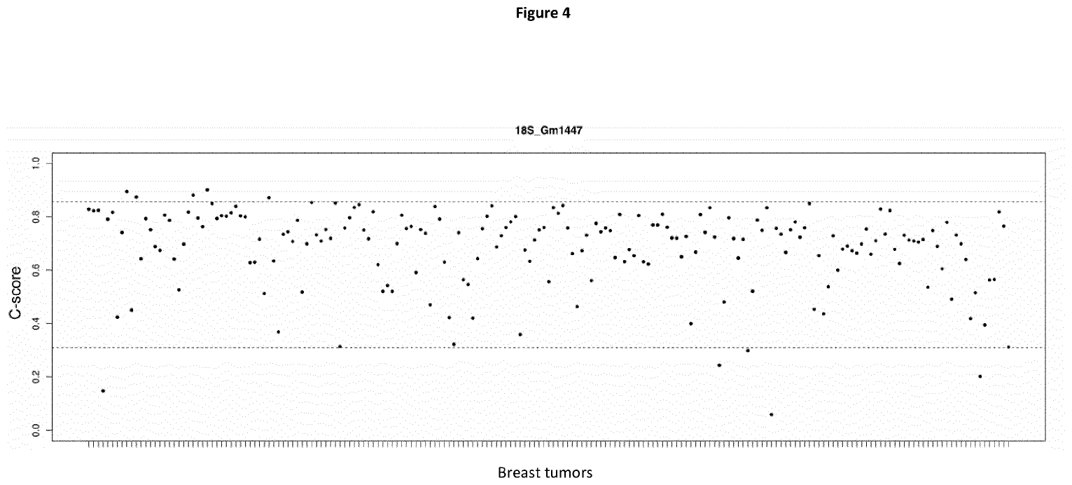

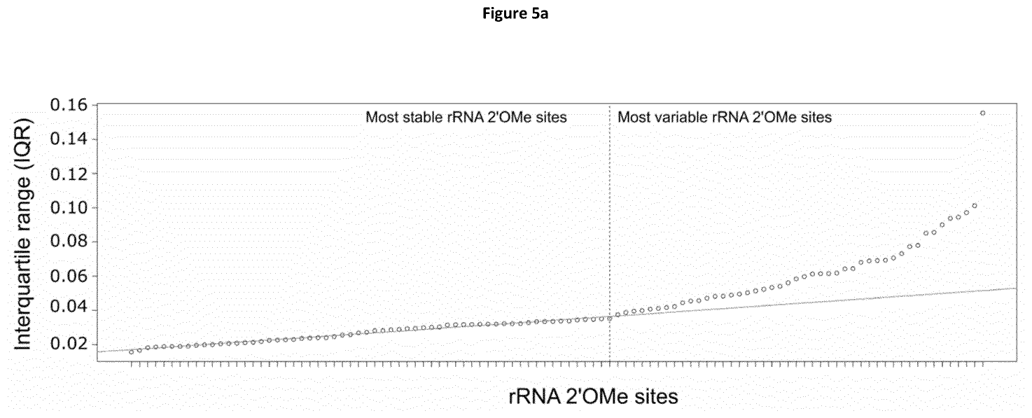

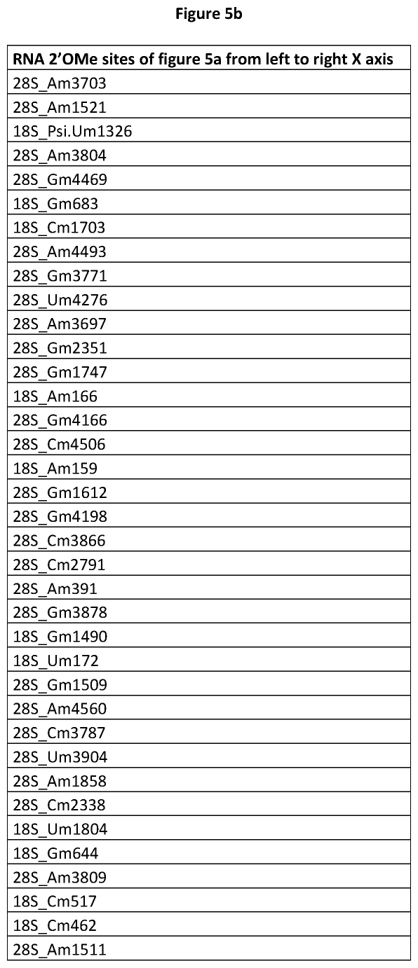

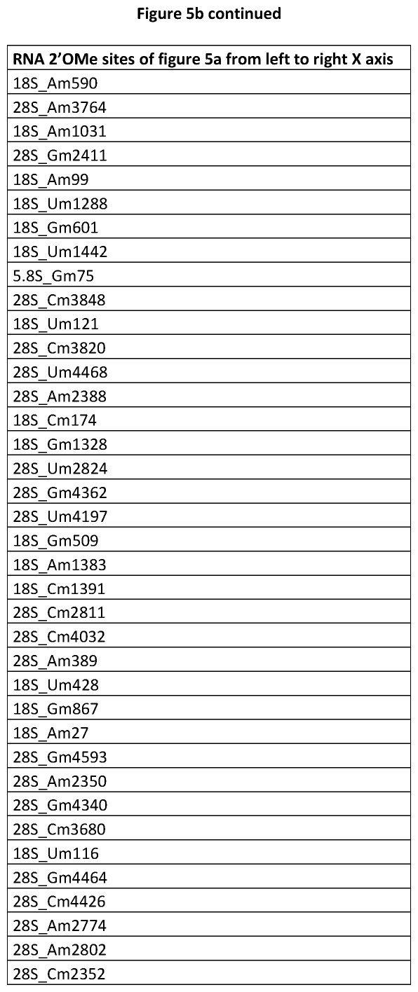

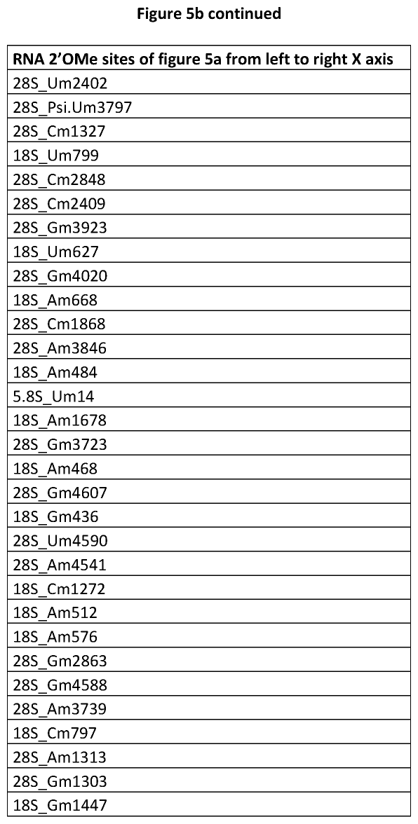

Hierarchical clustering also revealed that, fora given rRNA site, 2′O-methylation levels differ when comparing the 195 human tumours (i.e., inter-patient variability) (FIG. 2). We defined two classes of rRNA 2′O-methylated sites based on the variability of their 2′O-methylation level among patients (FIG. 3). The classification into these two classes was done empirically based on the intra-variability of the sites (in terms of interquartile range, IQR, at a given site) (FIGS. 4-5). The first class contains 60 rRNA 2′O-methylated sites (56.6% of all the rRNA 2′O-methylated positions), the levels of which exhibit low inter-patient variability between the 195 tumour samples, despite these series representing the full spectrum of breast cancer subtypes (FIGS. 2-3). Thereafter, they are termed “stable” sites since they display the most stable C-scores between the 195 tumours. The second class corresponds to a limited number of rRNA 2′O-methylated sites (46 sites, around 43.4% of the rRNA 2′O-methylated positions) that exhibit high inter-patient variability in their 2′O-methylation level (“variable” sites). Among them, the 28S-Am1313, 28S-Cm2352 and 28S-Gm3723 sites displayed important inter-patient variability, the C-scores ranging from 0 to >0.90 and a null C-score being observed in different tumours (FIG. 3). These data show that rRNA 2′O-methylation varies between human samples and demonstrate the co-existence of stable and variable classes of rRNA 2′O-methylated sites within human samples.

### Example 2—Comparison of Variant Sites in Different Cancer Types

### Materials and Methods

Human samples. A series of 6 samples issued from acute myeloid leukemia (AML) patients were used. Samples corresponded to CD34+ cells, which were purified from bone marrow and/or blood samples. Total RNA purification was performed as described by Bourdon et al. (12).

A series of 33 samples issued from splenic marginal zone lymphoma (SMZL) patients were used. SMZL corresponds to B-cell lymphoma. Total RNA purification was performed using the NucleoSpin Tripep kit (Macherey-Nalgen), as described by the supplier.

RiboMeth-seq analysis was applied, as described in Example 1 above.

### Results

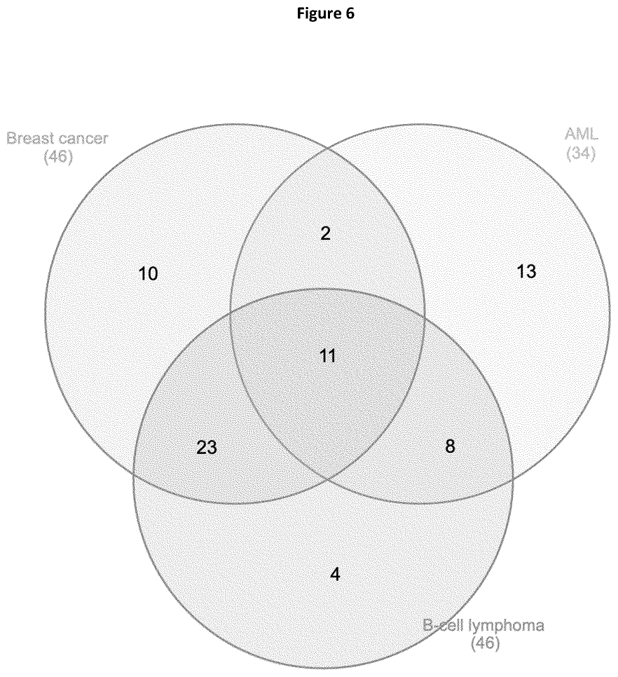

To identify common rRNA 2′Ome sites in different cancer types, rRNA 2′Ome level at the 106 2′Ome sites of rRNAs was first measured using RiboMeth—seq in a cohort of solid cancers (breast cancer, n=195) and two series of hematological cancers (AML, n=6; B-cell lymphoma, n=33). For each cancer type, the methylation status (i.e., stable vs variable) of each rRNA 2′Ome site was determined by analysing variability of the rRNA 2′Ome level among the tumour samples using IQR (as described in Example 1 above). Results are represented in FIG. 6.

It appeared that the number of variable sites is dependent upon the cancer type, breast cancer and B lymphoma being associated with 46 variable sites and AML to 33. It cannot be excluded that this difference may result from the smaller number of samples available for the AML cancer type. Then, the 3 lists of variable sites were confronted using Venn diagram to identify variable sites common to either the 3 cancer types, only two cancer types or specific to a particular one. This analysis identified 11 variable rRNA 2′Ome sites that are common to the 3 cancer types, from 2 to 23 variable rRNA 2′Ome sites that are common to two cancer types (AML-Breast cancer, n=2; AML-B lymphoma, n=8; Breast cancer-B lymphoma, n=23), and from 4 and 13 sites that are specific of each cancer type (AML, n=13; breast cancer, n=10; B-cell lymphoma, n=4).

### Example 3—Association of rRNA 2′O-Methylation Level With Biological and Clinical Characteristics

### Materials and Methods

Human samples. A cohort of 46 samples issued from brain samples was build. It was composed of 6 healthy donors and 40 glioma (grades III-IV) representing 3 different anatomopathological subtypes (13 oligodendroglioma, 13 astrocytoma and 14 glioblastoma). Total RNA purification was performed using the Maxwell RSC SimplyRNA Tissue Kit (Promega), as described by the supplier.

RiboMeth-seq analysis was applied, as described in Example 1 above.

Statistical analyses. Between-group comparisons were performed using Fisher's exact test for categorical data or Mann-Whitney test for quantitative data. Bonferroni or FDR (False Discovery Rate) correction methods were applied for multiple comparisons (P.adj). All P-values corresponded to two-tailed P-values. Significant association was considered when the adjusted P-value <0.05 and 4C-score (C-score condition 1−C-score condition 2)>0.05. Results were summarised using bubble plots where −log₁₀(P.adj) are represented with grey scale, absolute difference in C-score between the two conditions of interest by circle size.

Survival curves for overall survival (OS) with associated log-rank tests were generated using the Kaplan Meier method. OS corresponded to the timing from the date of diagnosis to either the date of death or last follow-up for censored patients. Survival median was estimated using the inverted Kaplan-Meier method. Unsupervised data analysis was performed using Principal Component Analysis (PCA) method, using the C-scores at individual rRNA 2′O-methylated sites. Correlation between the different dimensions of the principal component analysis reflecting different rRNA 2′O-methylation profiles and clinical data was performed using pearson correlation. All P-values corresponded to two-tailed P-values. Significant association was considered when the P-value <0.05, indicative that there is less than 5% probability that the null-hypothesis is truer. When P-value >0.05 but <0.1, although the null-hypothesis is rejected, the tendency of the data is still indicative of a putative biological event, and “borderline association” is used to qualify this trend. Statistical analyses and graphical representations were performed using either R v3.6.3 or GraphPad Prism v7.0a software (GraphPad Software, Inc).

### Results

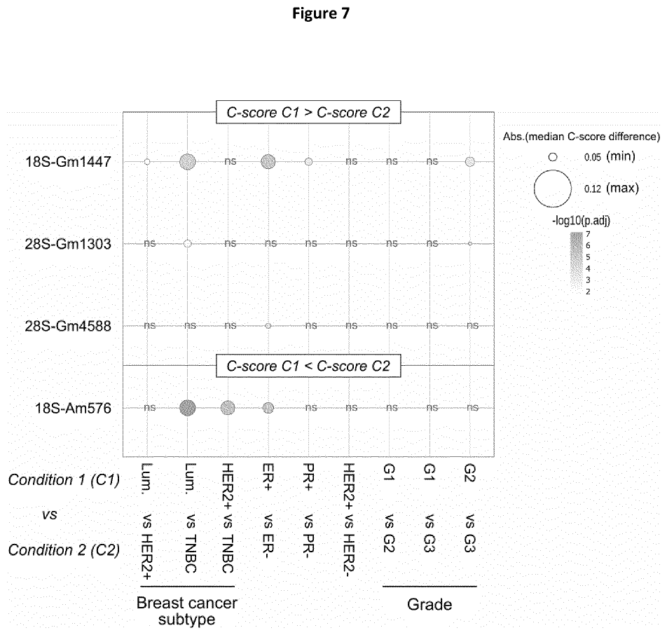

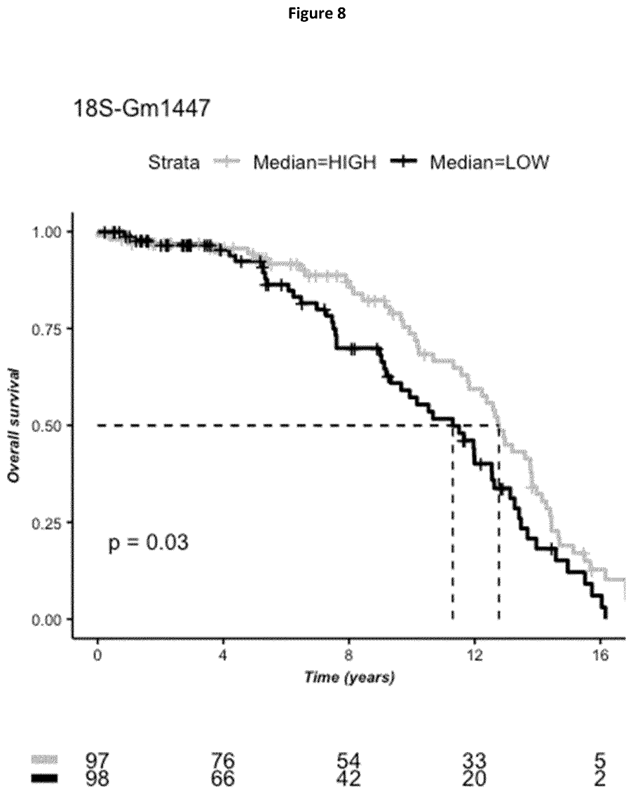

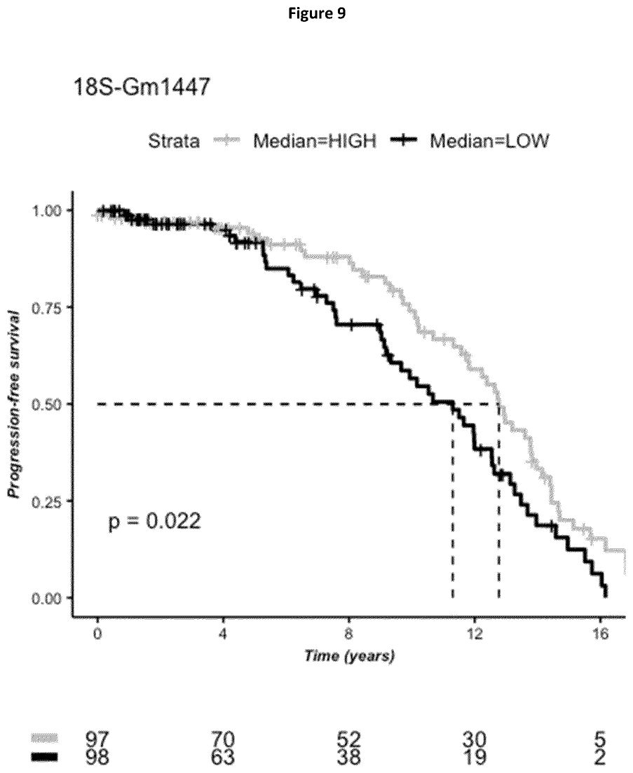

To assess the biological and clinical significance of the co-existence of two classes of rRNA 2′O-methylated positions displaying either the most “stable” 2′O-methylation level between human tumour samples or the most “variable” 2′O-methylation level, we evaluated the association between variations in C-score at the 106 rRNA 2′O-methylated positions and biological or clinical characteristics of primary breast tumours. Remarkably, among the stable rRNA 2′O-methylated sites, no association between change in C-score and well-known characteristics of breast cancers was observed (i.e., tumour grade, tumour size, lymph node involvement, hormonal/HER2 receptor status, breast cancer subtype or TP53 mutation). In contrast, we identified 4 variable rRNA 2′O-methylated sites (18S-Gm1447, 28S-Gm1303, 28S-Gm4588, 18S-Am576), the levels of which are significantly different between breast cancer subtypes, oestrogen and progesterone statuses, as well as tumour grades (FIG. 7). Moreover, it appears that the rRNA 2′O-methylation level at site 18S-Gm1447 is significantly associated with overall survival (P=0.03*, FIG. 8) and progression-free survival (P=0.022*, FIG. 9).

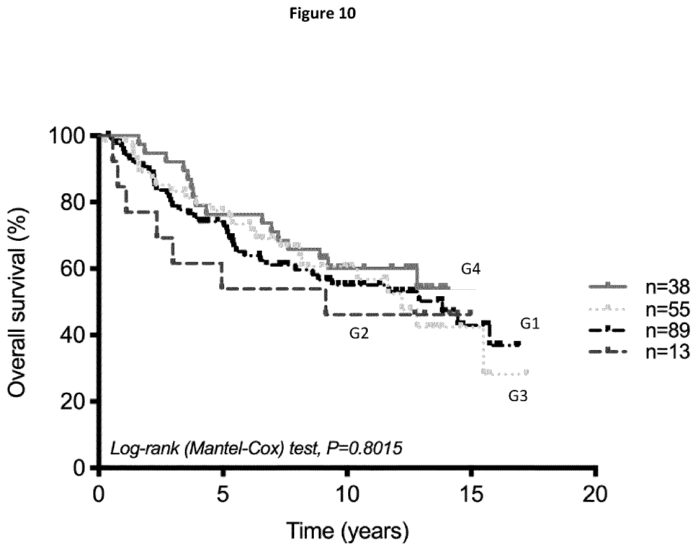

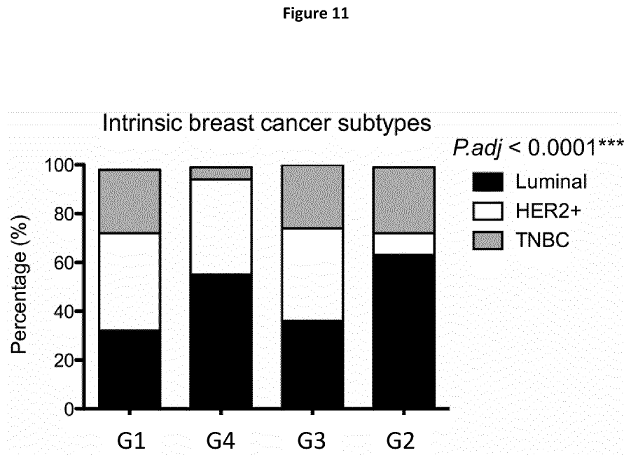

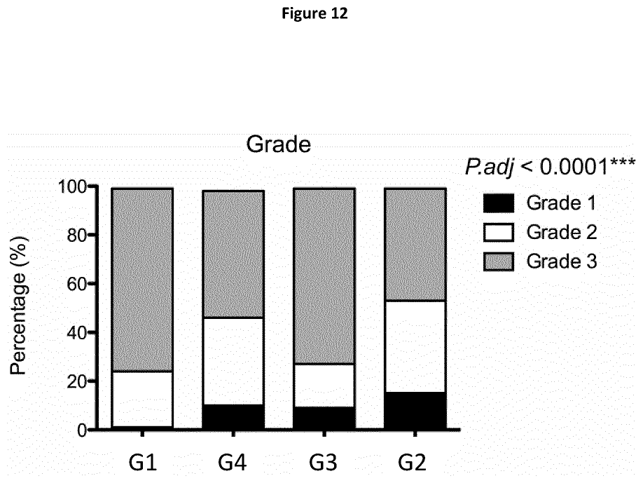

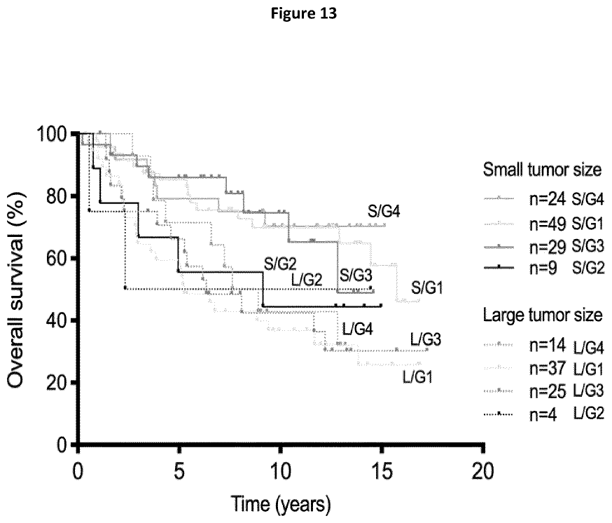

Secondly, we evaluated the association between breast cancer patients harboring tumours exhibiting similar rRNA 2′O-methylation profiles and biological/clinical characteristics of breast tumours. Hierarchical clustering and PCA methods identified 4 groups of breast cancer tumours based on their rRNA 2′O-methylation profiles (FIG. 2). Analysis of survival indicates that, although no significant association was observed, the patients carrying breast tumours characterized by distinct rRNA 2′O-methylation profile display distinguishable overall survival (FIG. 10). In particular the G2 group displays the poorest overall survival. This G2 group was enriched in TNBC and in tumours of high grade compared with the other groups (FIGS. 11 and 12). Regarding the fact that tumours in the G2 groups exhibited the lowest C-score at most of the rRNA 2′O-methylated sites (FIG. 2), these data suggest that global decrease in rRNA 2′O-methylation might be associated with breast tumour aggressiveness. Finally, clustering breast cancer tumours on the basis of their 2′O-methylation level at the 106 rRNA sites highlights at diagnosis, patients carrying small tumours size who display prognosis as poor as patients carrying large tumours size (FIG. 13). Indeed, patients carrying small tumour size exhibit significant different survival rates depending on the rRNA 2′O-methylation profile (P=0.0059**). Such data suggest that the rRNA 2′O-methylation profile could help in identifying patients with the poorest outcome although they carry small tumours that are generally associated with a low risk factor in breast cancer. Such patients may thus benefit from a more aggressive treatment protocol usually reserved to treat largest breast tumours.

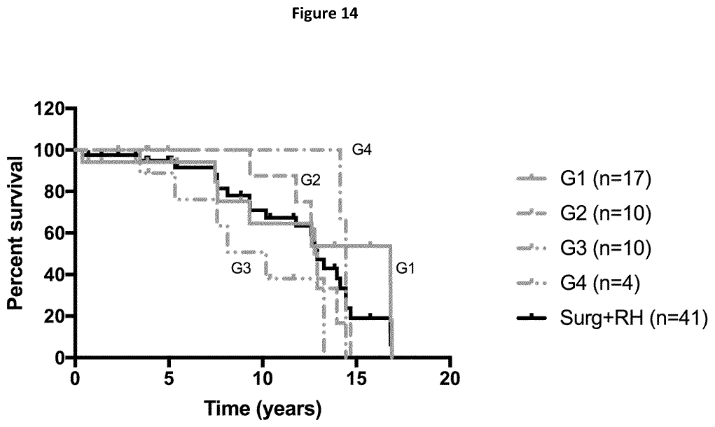

Finally, we determined whether rRNA 2′O-methylation profiles are differentially associated with response to treatment. Overall survival of breast cancer patients treated with surgery and combination of adjuvant radiotherapy/hormonotherapy was compared (FIG. 14). A trend has been observed suggesting that breast cancer patients show different clinical outcome depending on rRNA 2′O-methylation profile of their tumours (P=0.0665). Indeed, while patients carrying tumors with G2-rRNA 2′O-methylation profile display better outcome than the whole population, patients with tumours of G3-rRNA 2′O-methylation profile display a poorer outcome than the whole population. Thus, rRNA 2′O-methylation might be predictive of the benefit of a particular treatment regimen. Overall, these data indicate that 2′O-methylation levels at variable rRNA sites are associated with biological and clinical characteristics.

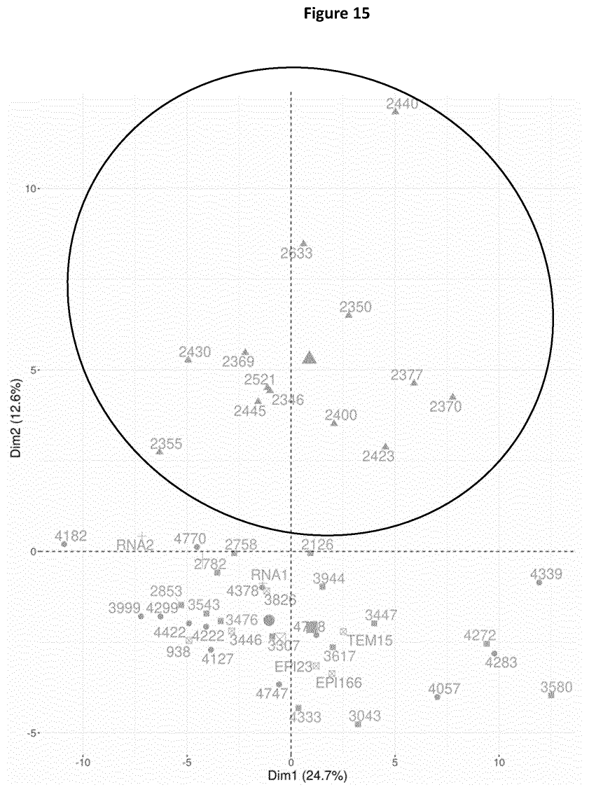

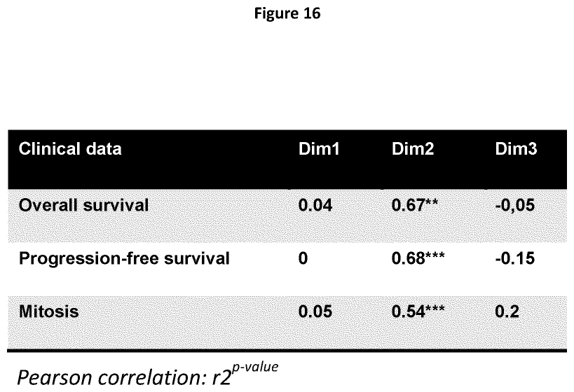

To determine whether the association between rRNA 2′O-methylation and clinical data occurs in another cancer type, we analyzed a cohort of 46 brain samples issued from healthy donors (n=6) and glioma patients (n=40). The glioma samples encompassed three high grade subtypes: oligodendroglioma (n=13), astrocytoma (n=13) and glioblastoma (n=14). PCA method allowed the clustering of two groups based on the rRNA 2′O-methylation profiles, one of them being composed exclusively of the 14 glioblastoma samples (FIG. 15). These data indicate that, like in breast cancers, rRNA 2′O-methylation profile could help in identifying patient with a particular glioma subtype. Association between glioma tumors displaying similar rRNA 2′O-methylation profiles and clinical characteristics corresponding to gold standard criteria to identified aggressive glioma tumors, were investigated using PCA dimension as rRNA 2′O-methylation group identifier (FIG. 16). A significant association was observed only between PCA dimension 2 and overall survival (P<0.001***), progression-free survival (P<0.0001***) and mitosis (P<0.0001***). It shows that groups of glioma tumors clustered on the basis of their rRNA 2′O-methylation profile and separated by dimension 2 associate with prognosis, the upper group being associated with the poorest prognosis (FIGS. 15 and 16). Overall, these data support the notion that 2′O-methylation levels at variable rRNA sites are associated with clinical characteristics, in different cancer types. Furthermore, rRNA 2′O-methylation might be used as biomarker of diagnosis (cancer subtype), prognosis (survival) and therapeutic prediction (treatment response).

### Example 4—Identifying Variable rRNA 2′OME Sites Can Help in Repositioning Ribosome-Targeting Drugs Such as Antibiotics

### Materials and Methods

Cell lines and cell viability. The epithelial and mesenchymal cellular models were kindly provided by Alain Puisieux's team (17). Briefly, the human epithelial mammary cell (hMECs) were used to derive EMT-related cellular models. The hMEC cell line corresponds to epithelial cells (Lonza) that were immortalized by lentiviral transduction allowing hTERT overexpression. The hMEC cell line was then transduced with lentiviral vector to induce stable over-expression of the murine EMT-TF ZEB1 and to promote EMT and thus generates the hMEC-ZEB1 mesenchymal cell line. The hMEC cellular models were maintained in DM EM-F12 (Gibo) supplemented with 0.5 μg/ml hydrocortisone, 10 ng/ml EGF, 0.3 μml insulin, 10% fetal calf serum, 100 μg/ml streptomycin and 100 units/ml of penicillin. Selective pressure was maintained with puromycin for hMEC-ZEB1. Cell viability was monitored in real-time using Incucyte Live-Cell analysis system (Statorius). Cells were plated in 96-well plates without selective pressure for 24 hrs before addition of the anisomycin antibiotic (range concentration 2-200 nM). DMSO was used as negative control.

### Results

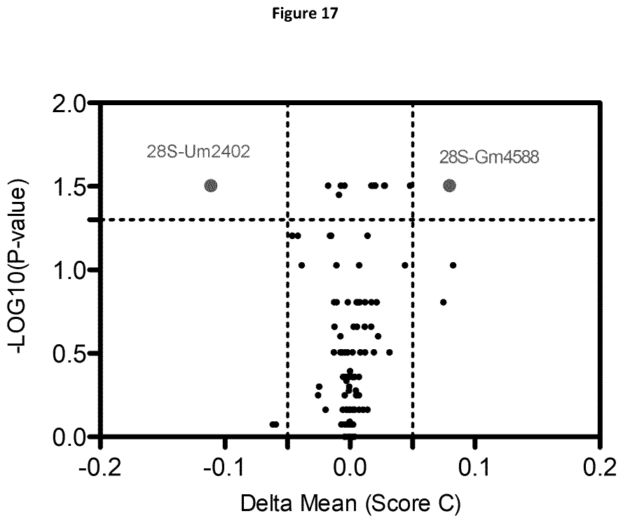

To make the proof of concept that identifying variable rRNA 2′Ome sites can help in repositioning ribosome-targeting drugs such as antibiotics, we first compare rRNA 2′Ome level at the 106 positions in epithelial hMEC cell line and in mesenchymal hMEC-ZEB1 one using RiboMeth-seq (n=5), so as to identify variable rRNA 2′Ome sites in mesenchymal cells compared to epithelial cells. Results are represented in FIG. 17.

A significant variation in rRNA 2′Ome level was observed for two sites (P-value <0.05*). It indeed appeared that rRNA 2′Ome level of 28S-Um2402 site significantly decreased in hMEC-ZEB1 mesenchymal cells compared to hMEC epithelial ones. In contrast, a significant increase in 2′Ome level at 28S-Gm4588 site was observed in hMEC-ZEB1 mesenchymal cells compared to hMEC epithelial ones.

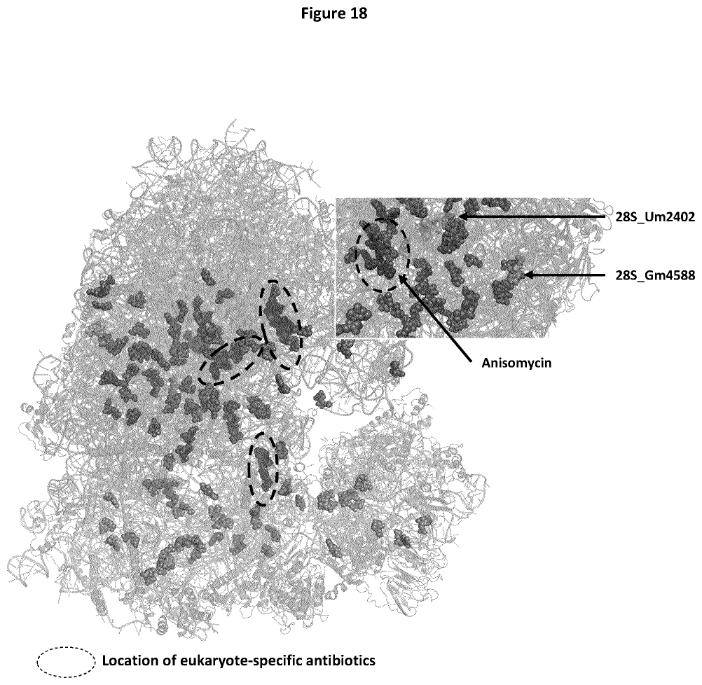

Then, to identify regions of the mesenchymal ribosomes affected by the changes in rRNA 2′Ome level, we located all the 2′Ome sites of the 3 rRNAs on the available human structure of the ribosome resolved by Cryo-EM (PDB, AUG0) (14). Results are represented in FIG. 18.

Nucleotide sites are shown as spheroid dots. In addition, we located the binding pockets of well-known eukaryote-specific antibiotics (dotted circles). We observed that the variable sites, 28S-Um2402 and 28S-Gm4588, the 2′Ome level of which varies between mesenchymal and epithelial cells, are in the closed vicinity of the binding pocket of the anisomycin antibiotic. Interestingly, the three-dimensional visualization indicates that the 28S-Gm4588 is closer from the binding pocket of the anisomycin antibiotic than the 28S-Um2402, indicating that rRNA 2′Ome level of 28S-Gm4588 might affect more importantly the anisomycin binding than 28S-Um2402. This visualization using three-dimensional ribosome structure allows identification of a single region of the ribosome affected by variable site, this region being closed to the one binding by a particular eukaryote-specific antibiotic.

Finally, to demonstrate that determining variable 2′Ome sites is useful to select appropriate ribosome-targeting drugs, the hMEC epithelial and hMEC-ZEB1 mesenchymal cells were treated with increasing concentration of anisomycin to determine a potential difference in sensitivity to this antibiotic.

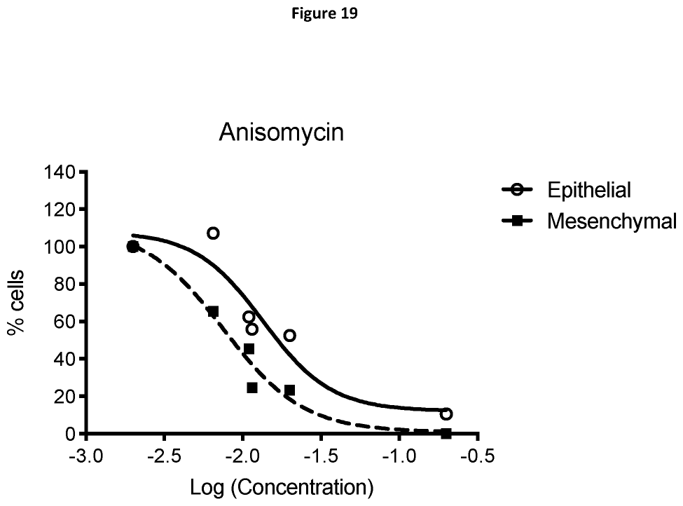

Cell viability was monitored using real-time monitoring system and no change in cell morphology was observed, either for the epithelial or the mesenchymal cells. Results are summarized in FIG. 19.

At day 6, difference in cell viability was determined using cell confluence as a read-out. We observed that the hMEC-ZEB1 mesenchymal cells are more sensitive to anisomycin treatment that the hMEC epithelial cells. These data suggest that targeting ribosomal region displaying rRNA sites whose 2′Ome level varies, with an antibiotic binding specifically to this ribosome region results in decreased cell viability.

### BIBLIOGRAPHIC REFERENCES

1. Marcel, V., Ghayad, S. E., Belin, S., Therizols, G., Morel, A. P., Solano-Gonzàlez, E., Vendrell, J. A., Hacot, S., Mertani, H. C., Albaret, M. A., et al. (2013) P53 Acts as a Safeguard of Translational Control by Regulating Fibrillarin and rRNA Methylation in Cancer. Cancer Cell, 24, 318-330.
2. Erales, J., Marchand, V., Panthu, B., Gillotf, S., Belin, S., Ghayad, S. E., Garcia, M., Laforets, F., Marcel, V., et al. (2017) Evidence for rRNA 2 {′}-O-methylation plasticity: Control of intrinsic translational capabilities of human ribosomes. Proc. Natl. Acad. Sci., 10.1073/PNAS.1707674114.
3. Helm, M. and Motorin, Y. (2017) Detecting RNA modifications in the epitranscriptome: predict and validate. Nat. Rev. Genet., 18, 275-291.
4. Decatur, W. A. and Fournier, M. J. (2002) rRNA modifications and ribosome function. Trends Biochem Sci, 27, 344-351.
5. Lo Monaco, P., Marcel, V., Diaz, J. J. and Catez, F. (2018) 2′-O-methylation of ribosomal RNA: Towards an epitranscriptomic control of translation? Biomolecules, 8.
6. Esteller, M. (2011) Non-coding RNAs in human disease. Nat Rev Genet, 12, 861-874.
7. Marchand, V., Blanloeil-Oillo, F., Helm, M. and Motorin, Y. (2016) Illumina-based RiboMethSeq approach for mapping of 2′-O-Me residues in RNA. Nucleic Acids Res., 44.
8. Birkedal, U., Christensen-Dalsgaard, M., Krogh, N., Sabarinathan, R., Gorodkin, J. and Nielsen, H. (2015) Profiling of ribose methylations in RNA by high-throughput sequencing. Angew. Chemie—Int. Ed., 54, 451-455.
9. Krogh, N., Nielsen, H., 2019. Sequencing-based methods for detection and quantitation of ribose methylations in RNA. Methods 156, 5-15.
10. Belin, S., Beghin, A., Solano-Gonzàlez, E., Bezin, L., Brunet-Manquat, S., Textoris, J., Prats, A.-C., Mertani, H. C., Dumontet, C. and Diaz, J.-J. (2009) Dysregulation of Ribosome Biogenesis and Translational Capacity Is Associated with Tumor Progression of Human Breast Cancer Cells. PLoS One, 4, e7147.
11. Ayadi, L., Galvanin, A., Pichot, F., Marchand, V., Motorin, Y., (2018). BBA—Gene Regulatory Mechanisms. BBA—Gene Regulatory Mechanisms 1-17.
12. Bourdon, J.-C., Khoury, M. P., Diot, A., Baker, L., Fernandes, K., Aoubala, M., Quinlan, P., Purdie, C. A., Jordan, L. B., Prats, A.-C., et al. (2011) p53 mutant breast cancer patients expressing p53$γ$ have as good a prognosis as wild-type p53 breast cancer patients. Breast Cancer Res., 13, R7.
13. Marchand, V., Ayadi, L., El Hajj, A., Blanloeil-Oillo, F., Helm, M. and Motorin, Y. (2017) High-Throughput Mapping of 2′-O-Me Residues in RNA Using Next-Generation Sequencing (Illumina RiboMethSeq Protocol). In. Springer New York, New York, NY, pp. 171-187.
14. Khatter, H., Myasnikov, A. G., Natchiar, S. K. and Klaholz, B. P. (2015) Structure of the human 80S ribosome. Nature, 520,640-645.
15. Natchiar, S. K., Myasnikov, A. G., Kratzat, H., Hazemann, I. and Klaholz, B. P. (2017) Visualization of chemical modifications in the human 80S ribosome structure. Nature, 551, 472-477.
16. Natchiar, S., Myasnikov, A., Hazemann, I. and Klaholz, B. (2018) Visualizing the Role of 2′-OH rRNA Methylations in the Human Ribosome Structure. Biomolecules, 8, 125.
17. Morel, A.-P., Hinkal, G. W., Thomas, C., Fauvet, F., Courtois-Cox, S., Wierinckx, A., Devouassoux-Shisheboran, M., Treilleux, I., Tissier, A., Gras, B., Pourchet, J., Puisieux, I., Browne, G. J., Spicer, D. B., Lachuer, J., Ansieau, S., Puisieux, A. (2012) EMT Inducers Catalyze Malignant Transformation of Mammary Epithelial Cells and Drive Tumorigenesis towards Claudin-Low Tumors in Transgenic Mice. PLoS Genet 8, e1002723.
18. Truitt, M. L, Ruggero, D. (2016) New frontiers in translational control of the cancer genome. Nat Rev Cancer 16, 288-304.

1. A method for identifying potentially relevant markers in cancer diagnosis, prognosis and/or estimation of treatment benefit and/or therapy comprising:
a) measuring the 2′O-ribose methylation level of the 2′O-ribose methylation positions of ribosomal RNAs (rRNAs) in biological samples from a representative population of patients suffering from a cancer,
b) assessing the individual methylation status for each 2′O-ribose methylation positions by determining the variability of the 2′O-ribose methylation level thus measured for each 2′O-ribose methylation position between each sample of patients from the representative population, and
c) selecting the set of 2′O-ribose methylation positions for which the individual methylation status is a “variable status”.

2. The method of claim_1, wherein the 2′O-ribose methylation level of the 2′O-ribose methylation positions is determined by a C-score calculated using RiboMeth-seq method.

3. The method of claim 1, wherein step b) is carried out by:
b1) determining the variability of the 2′O-ribose methylation level measured for each 2′O-ribose methylation position between each sample of patients from the representative population, and
b2) determining for each 2′O-ribose methylation position the methylation status by comparing the variability of all 2′O-ribose methylation positions among the representative population.

4. The method of claim 1, wherein the variability of the 2′O-ribose methylation level for each 2′O-ribose methylation position, which is determined for assessing the methylation status, is determined by a statistical approach which allows the comparison of the variability of each rRNA 2′Ome level between each sample of patients from the representative population for each 2′Ome position independently, and then the methylation status of each 2′O-ribose methylation position is determined regarding a threshold corresponding to the minimal value of the variability of the 2′O-ribose methylation level at one particular position, that shows a deviation from the values of the variability of the 2′O-ribose methylation level of the other 2′O-ribose methylation positions among the representative population.

5. The method of claim 1, wherein the set selected at step c) contains from 5 to 50 2′O-ribose methylation positions among the 106 2′O-ribose methylation positions of rRNAs whatever the cancer the patients are suffering from.

6. The method of claim 5, wherein the set selected at step c) contains the following 34 positions among the 106 2′O-ribose methylation positions of rRNAs:
18S_Am27; 18S_Am468; 18S_Am484; 18S_Am512; 18S_Cm1272; 18S_Cm797;
18S_Gm1447; 18S_Gm436; 18S_Gm867; 18S_Um428; 18S_Um627; 18S_Um799;
28S_Am1326; 28S_Am2787; 28S_Am3867; 28S_Am4571; 28S_Cm1340; 28S_Cm1881;
28S_Cm2409; 28S_Cm3701; 28S_Gm1316; 28S_Gm2876; 28S_Gm3744; 28S_Gm3944;
28S_Gm4042; 28S_Gm4370; 28S_Gm4494; 28S_Gm4618; 28S_Gm4623; 28S_Gm4637;
28S_Psi-Um3818; 28S_Um2415; 28S_Um4620; 5.8S_Um14.

7. The method of claim 1, wherein patients are suffering from breast cancer, and the set of 2′O-ribose methylation positions selected at step c) contains from 40 to 50 2′O-methylation positions among the 106 2′O-ribose methylation positions of rRNAs.

8. The method of claim 1, wherein patients are suffering from glioma, and the set of 2′O-ribose methylation positions selected at step c) contains from 30 to 40 2′O-methylation positions among the 106 2′O-ribose methylation positions of rRNAs.

9. A method for determining the prognostic of a patient suffering from cancer irrespective of the treatment, comprising:
a) measuring the 2′O-ribose methylation level of the 2′O-ribose methylation positions of ribosomal RNAs (rRNAs) in a sample from a patient suffering from a cancer of whom the prognostic is to be determined, called “tested patient”,
b) comparing of the 2′O-ribose methylation level of the 2′O-ribose methylation positions of ribosomal RNAs (rRNAs) measured with that of a representative population of patients suffering from the same cancer than the “tested patient” and for whom the clinical outcome is known, and
c) determining the prognostic of the “tested patient” by identifying the one corresponding to the group of patients from the representative population which has the 2′O-ribose methylation level measures closer to that of the “tested patient”.

10. The method of claim 9, wherein the comparison at step b) is carried out using a 2′O-ribose methylation position-by-position analysis or using a whole profile analysis of all the 2′O-ribose methylation positions.

11. The method of claim 9, wherein the patient is suffering from a breast cancer and wherein the comparison of the 2′O-ribose methylation levels is carried out with a 2′O-ribose methylation position-by-position analysis limited to at least one of the four 2′O-ribose methylation positions: 18S-Gm1447; 28S_Gm1316; 28S_Gm4618 and 18S-Am576.

12. A method for estimating the benefit of a treatment in a patient suffering from cancer, comprising:
a) measuring the 2′O-ribose methylation level of the 2′O-ribose methylation positions of ribosomal RNAs (rRNAs) in a sample from a patient suffering from a cancer for whom the benefit of a specific treatment is to be determined, called “tested patient”,
b) comparing of the 2′O-ribose methylation level of the 2′O-ribose methylation positions of ribosomal RNAs (rRNAs) measured with that of a representative population of patients suffering from the same cancer than the “tested patient” and for whom the benefit of one or more specific treatments is known, and
c) determining the expected benefit of the specific treatment for the “tested patient” by selecting the one corresponding to the group of patients from the representative population which has the 2′O-ribose methylation level measures closer to that of the “tested patient”.

13. The method of claim 12, wherein the comparison is carried out using a 2′O-ribose methylation position-by-position analysis or using a whole profile analysis of all the 2′O-ribose methylation positions.

14. A method for selecting one or more therapeutic drug(s) targeting ribosomes, useful for treating cancers, comprising:
determining the target region(s) on the ribosome corresponding to ribosomal region(s) which comprise one or more 2′O-ribose methylation positions, the 2′O-ribose methylation level of which being known to be associated with cancer, and
identifying one or more ribosome-targeting drugs, including antibiotics, directed to said target region(s).

15. The method of claim 14, wherein the 2′O-ribose methylation positions associated with cancer are identified by:
a) measuring the 2′O-ribose methylation level of the 2′O-ribose methylation positions of ribosomal RNAs (rRNAs) in biological samples from a representative population of patients suffering from a cancer,
b) assessing the individual methylation status for each 2′O-ribose methylation positions by determining the variability of the 2′O-ribose methylation level thus measured for each 2′O-ribose methylation positions between each sample of patients from the representative population, and
c) selecting the set of 2′O-ribose methylation positions for which the individual methylation status is a “variable status”.

16. The method of claim 14, wherein the 2′O-ribose methylation positions associated with cancer are identified by:
a) measuring the 2′O-ribose methylation level of the 2′O-ribose methylation positions of ribosomal RNAs (rRNAs) in biological samples from a representative population of patients suffering from a cancer and in biological samples from a control population of patients not suffering from a cancer,
b) assessing the individual methylation status for each 2′O-ribose methylation positions by determining the variability of the 2′O-ribose methylation level thus measured for each 2′O-ribose methylation positions between each sample of patients from the two populations, and
c) selecting the set of 2′O-ribose methylation positions for which the individual methylation status is a “variable status” in the representative population of patients suffering from a cancer and not in the population of patients not suffering from a cancer.

17. The method of claim 14, wherein:
a. the target region(s) on the ribosome are determined by the following approach:
a1. Analysis of the 2′Ome of patients
a2. Identification of variable site(s) predictive of a particular molecular cancer subtype/poor prognosis and/or no or poor benefit of an available treatment and/or tumoral phenotypic traits, which correspond to the 2′O-ribose methylation positions, the 2′O-ribose methylation level of which is known to be associated with cancer,
a3. Location of this/these variable site(s) on the structure of the ribosome,
a4. Determination of the distance between this/these variable site(s) and the location of pocket binding of ribosome-targeting drugs on the structure of the ribosome; and
b. the one or more ribosome-targeting drugs are identified by identifying the one or those allowing the inhibition of ribosome activity and whose pocket binding within the ribosome shows the smallest and equal distance to 1 or more variable site(s) predictive of a particular molecular cancer subtype/poor prognosis and/or no or poor benefit of a treatment and/or tumoral phenotypic traits.

18. The method of claim 7, wherein the set of 2′O-ribose methylation positions selected at step c) contains the following 46 2′O-ribose methylation positions among the 106 2′O-ribose methylation positions of rRNAs:
18S_Am1678; 18S_Am27; 18S_Am468; 18S_Am484; 18S_Am512; 18S_Am576;
18S_Am668; 18S_Cm1272; 18S_Cm797; 18S_Gm1447; 18S_Gm436; 18S_Gm867;
18S_Um116; 18S_Um428; 18S_Um627; 18S_Um799; 28S_Am1326; 28S_Am2363;
28S_Am2787; 28S_Am2815; 28S_Am3760; 28S_Am3867; 28S_Am398; 28S_Am4571;
28S_Cm1340; 28S_Cm1881; 28S_Cm2365; 28S_Cm2409; 28S_Cm2861; 28S_Cm3701;
28S_Cm4054; 28S_Cm4456; 28S_Gm1316; 28S_Gm2876; 28S_Gm3744; 28S_Gm3944;
28S_Gm4042; 28S_Gm4370; 28S_Gm4494; 28S_Gm4618; 28S_Gm4623; 28S_Gm4637;
28S_Psi-Um3818; 28S_Um2415; 28S_Um4620; 5.8S_Um14.

19. The method of claim 8, wherein the set of 2′O-ribose methylation positions selected at step c) contains the following 32 2′O-ribose methylation positions among the 106 2′O-ribose methylation positions of rRNAs:
18S_Am576; 18S_Cm1272; 18S_Cm174; 18S_Gm1447; 18S_Um116; 28S_Gm1760;
28S_Am2363; 28S_Am2401; 28S_Am3760; 28S_Am3785; 28S_Am3825; 28S_Am3867;
28S_Am400; 28S_Am4523; 28S_Am4571; 28S_Am4590; 28S_Cm1340; 28S_Cm2824;
28S_Cm2861; 28S_Cm4536; 28S_Gm2876; 28S_Gm3792; 28S_Gm3944; 28S_Gm4042;
28S_Gm4494; 28S_Gm4499; 28S_Gm4618; 28S_Gm4623; 28S_Gm4637; 28S_Um2415;
28S_Um2837; 28S_Um4620.
